(19) United States
(12) Patent Application Publication
(10) Pub. No.: US 2024/0279231 A1
(43) Pub. Date: Aug. 22, 2024

(54) MAP4K4 INHIBITORS AND METHODS OF SYNTHESIS AND USE THEREOF

(71) Applicant: The Board of Regents of the University of Oklahoma, Norman, OK (US)

(72) Inventors: Vibhudutta Awasthi, Edmond, OK (US); Venkateswararao Eeda, Oklahoma City, OK (US)

(73) Assignee: The Board of Regents of the University of Oklahoma, Norman, OK (US)

(21) Appl. No.: 18/562,343

(22) PCT Filed: May 20, 2022
(86) PCT No.: PCT/US2022/030340

§ 371 Date: Nov. 20, 2023

Related U.S. Application Data
(60) Provisional application No. 63/191,243, filed on May 20, 2021.

(51) Int. Cl. C07D 487/04; C07F 9/6561; C07H 19/14

(52) U.S. Cl. CPC C07D 487/04; C07F 9/6561; C07H 19/14

(57) ABSTRACT

The compound DMX-5804, IUPAC name 5-(4-(2-methoxyethoxy)phenyl)-7-phenyl-3,4a,7,7a-tetrahydro-4H-pyrrolo[2,3-d]pyrimidin-4-one, is a potent and selective inhibitor of mitogen-activated protein kinase kinase kinase kinase-4 (MAP4K4). The present disclosure describes a scalable and practical synthesis process for making DMX-5804. The process (1) doesn't rely on transition metals, (2) has reduced duration of reactions, (3) is streamlined with significantly improved yields using low cost raw materials, (4) includes no microwave-assisted reactions, (5) does not require column purification, and (6) generally results in crystalized products having over a 95% purity in each step. The present disclosure also describes DMX-5804 analogs and derivatives for use in inhibiting MAP4K4, and scalable and practical processes for making such DMX-5804 analogs.

### CROSS-REFERENCE TO RELATED APPLICATIONS

This application claims the benefit of U.S. Provisional Application No. 63/191,243, filed May 20, 2021, the disclosure of which is expressly incorporated herein by reference in its entirety.

### BACKGROUND

Mitogen-activated protein kinase kinase kinase kinase-4 (MAP4K4), also known as HGK (hematopoietic progenitor kinase/germinal center kinase-like kinase) or NIK (Nck interacting kinase, the mouse ortholog), is a serine/thereonine kinase in the MAPK pathway. It plays an essential role in signal transduction by modulating gene transcription in the nucleus in response to changes in the cell environment. It belongs to the mammalian family of Ste20 protein kinases, with a molecular target of TGFβ-activated kinase-1 (TAK1 or MAP3K7) which is a key activator of JNK pathway. Recent work on MAP4K4 has indicated that it can be targeted for the treatment of cancer. Because of its relatively high expression in the brain and the crucial role of downstream p38 MAPK and JNK signaling in apoptosis, studies have also reported beneficial role of MAP4K4 inhibition in neuronal recovery post-injury. MAP4K4 signaling has also been reported to participate in myocardial infarction (MI). Myocardial MAP4K4 is activated in end-stage heart failure regardless of cause, i.e., dilation, hypertrophism, ischemia, and anthracycline-induced cardiomyopathy. Moreover, MAP4K4 also plays a defining role in pathologies that can be precursors to MI. For example, MAP4K4 expression and/or activity is increased in the aortas of mice and humans with atherosclerosis, and reduction of endothelial MAP4K4 expression ameliorated atherosclerotic lesion development and inflammatory signaling in a mouse model.

Given that MAP4K4 is only recently recognized for its regulatory role in myocardial injury and tissue recovery, research on the development of its small molecule inhibitors is relatively nascent. Oxidative stress-induced cardiomyocyte death has been demonstrated to require MAP4K4 activation and its pharmacologic inhibition by the novel molecule DMX-5804 reduces cardiomyocyte death both in vitro and in vivo (Fiedler, L. R., et al., MAP4K4 Inhibition Promotes Survival of Human Stem Cell-Derived Cardiomyocytes and Reduces Infarct Size In Vivo. Cell Stem Cell, 2019. 24(4): p. 579-591 e12). Using human stem cells-derived cardiomyocytes as a platform for target validation and compound development, two potent inhibitors of MAP4K4 viz 5,7-diphenyl-3,7-dihydro-4H-pyrrolo[2,3-d]pyrimidin-4-one (F1386-0303, IC₅₀ 34 nm), and 5-(4-(2-methoxyethoxy)phenyl)-7-phenyl-3,4a,7,7a-tetrahydro-4H-pyrrolo[2,3-d]pyrimidin-4-one (DMX-5804, IC₅₀ 3 nm) were identified. Administration of DMX-5804 in a mouse infarct model resulted in significant reduction of infarct size in a mouse model. DMX-5804 also exhibited better bioavailability in vivo than F1386-0303.

Large-scale production of DMX-5804 will be needed for clinical use. It is to providing, in one embodiment of the present disclosure, an easy, practical, scalable, high-yielding procedure for synthesis of crystalline DMX-5804. The present disclosure also provides an easy route for synthesizing derivatives and analogs of DMX-5804 for use as inhibitors of MAP4K4, and identifies a number of such derivatives and analogs.

### BRIEF DESCRIPTION OF THE DRAWINGS

FIG. 1 shows chemical structures of compounds 5,7-diphenyl-3,7-dihydro-4H-pyrrolo[2,3-d]pyrimidin-4-one (F1386-0303) and 5-(4-(2-methoxyethoxy)phenyl)-7-phenyl-3,4a,7,7a-tetrahydro-4H-pyrrolo[2,3-d]pyrimidin-4-one (DMX-5804), small molecule inhibitors of mitogen-activated protein kinase kinase kinase kinase-4 (MAP4K4).

FIG. 2 shows a high performance liquid chromatography (HPLC) profile of DMX-5804.

FIGS. 3A-3C shows the MAP4K4 inhibitory activity of DMX-5804 (FIG. 3A), compound 7 (FIG. 3B), and compound 9 (FIG. 3C).

FIG. 4 shows as Scheme I the original synthesis route for DMX-5804 (Fiedler et al., 2019).

FIG. 5 shows as Scheme II a novel synthesis route for DMX-5804.

FIG. 6 shows as Scheme III a novel synthesis route for derivatives and analogs of DMX-5804.

FIG. 7 shows as Scheme IV a novel alternate synthesis route for derivatives and analogs of DMX-5804. R¹-R⁴, V¹ and V² are those chemical Formulas IV and V.

FIG. 8 shows representative examples of DMX-5804 analogs.

FIG. 9 shows additional representative examples of DMX-5804 analogs.

FIG. 10 shows additional representative examples of DMX-5804 analogs.

FIG. 11 shows a generic chemical structure (Formula IV) of DMX-5804 derivatives and analogs which are intended for use as MAP4K4 inhibitors according to the methods of the present disclosure.

FIG. 12 shows a generic chemical structure (Formula V) of DMX-5804 derivatives and analogs which are intended for use as MAP4K4 inhibitors according to the methods of the present disclosure.

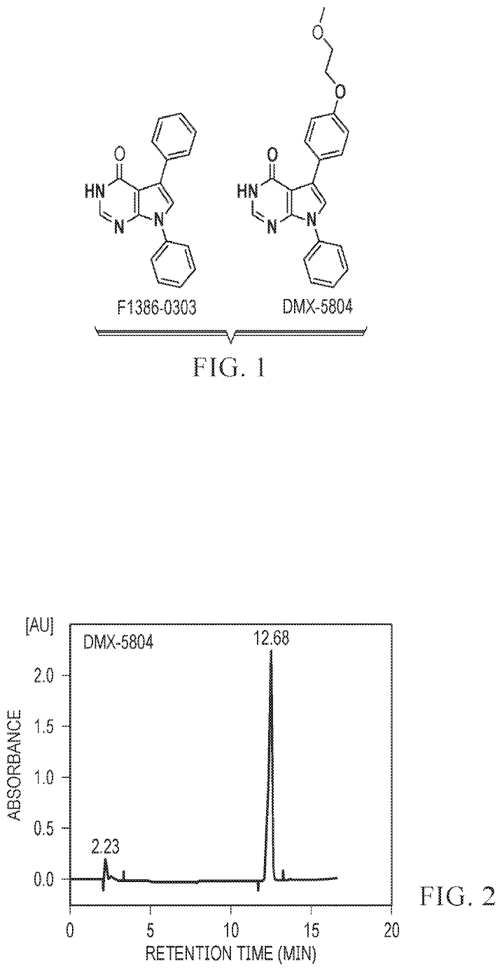

### DETAILED DESCRIPTION

Disclosed herein is a scalable and practical synthesis process for making and using the compound DMX-5804, IUPAC name 5-(4-(2-methoxyethoxy)phenyl)-7-phenyl-3,4a,7,7a-tetrahydro-4H-pyrrolo[2,3-d]pyrimidin-4-one, and derivatives thereof. DMX-5804 is a potent and selective inhibitor of mitogen-activated protein kinase kinase kinase kinase-4 (MAP4K4). The new process affords quantitative yields of DMX-5804 in a reproducible manner. Furthermore, the process (i) doesn't rely on transition metal catalysts, anhydrous solvents, or commercially unavailable borate intermediates, (ii) has reduced duration of reactions, (iii) is streamlined with significantly improved yields using low cost raw materials, (iv) includes no microwave-assisted reaction steps, (v) does not require column purification of intermediates or HPLC purification of the final product (which are required steps in the conventional method of synthesis of Fiedler et al.), and (vi) results in crystalized products having over a 95% purity in each step. The disclosure also provides an easy route for synthesizing derivatives and analogs of DMX-5804 for use as inhibitors of MAP4K4. Non-limiting examples of various DMX-5804 analogs intended for use as inhibitors of MAP4K4 are described. In certain embodiments, the DMX-5804 and analogs and derivatives thereof may be linked to one or more carbohydrate moieties to form a glycoconjugate.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the compounds, compositions, and methods of the present disclosure have been described in terms of particular (but non-limiting) embodiments, it will be apparent to those of skill in the art that variations may be applied to the compounds, compositions, and methods, and in the steps or in the sequence of steps of the methods described herein without departing from the concept, spirit, and scope of the inventive concepts.

All patents, patent applications, and non-patent publications including published articles mentioned in the specification or referenced in any portion of this application, including but not limited to U.S. Provisional Application No. 63/191,243, filed May 20, 2021, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities, and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are for purposes of illustration only and are to be construed as non-limiting of the claims.

Use of the word “we,” “us,” and/or “our” as a pronoun in the present disclosure refers generally to laboratory personnel, technicians, or other contributors who assisted in laboratory procedures and data collection and is not intended to represent an inventorship role by said laboratory personnel, technicians, or other contributors in any subject matter disclosed herein.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

Where used herein the term “active agent” refers to a compound or composition having a biological activity as described herein. “Biological activity” refers to a compound's ability to modify the physiological system of an organism without reference to how the compound has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Certain of the disclosed compounds may exist in various stereoisomeric forms. Stereoisomers are compounds that differ only in their spatial arrangement. Enantiomers are pairs of stereoisomers that are non-superimposable mirror images of one another, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. Compounds of the present disclosure may contain one or more asymmetrically-substituted carbon or nitrogen atoms and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### Chemical Group Definitions

When used in the context of a chemical group: “hydrogen” is —H; “deuterium” is -D; “hydroxy” means —OH; “borane” is —B; “oxo” is ═O; “carbonyl” is —C(═O)—; “carboxy” is —C(═O)OH (also written as —COOH or —CO₂H); the term “halogen” includes fluoro (fluorine, F), chloro (chlorine, Cl), bromo (bromine, Br), and iodo (iodine, I), “halo” means independently —F, —Cl, —Br or —I; “amino” is —NH₂; “hydroxyamino” is —NHOH; “nitro” is —NO₂; imino is ═NH; “cyano” is —CN; “isocyanate” is —N═C═O; “azido” is —N₃; in a monovalent context “phosphate” is —OP(O)(OH)₂ or a deprotonated form thereof; in a divalent context “phosphate” is —OP(O)(OH)O— or a deprotonated form thereof; “mercapto” is —SH; “thio” is ═S; “sulfonyl” is —S(O)₂—; and “sulfinyl” is —S(O)—.

In the context of chemical formulas, the symbol “-” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, the formula

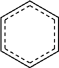

covers, for example,

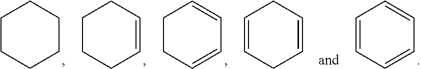

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

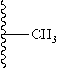

for methyl) indicates a point o attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

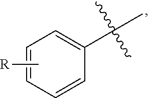

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

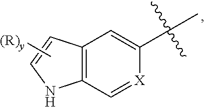

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “Cₙ” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤s)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “C₅ olefin”, “C₅-olefin”, “olefin_{(C5)}”, and “olefin_{C5}” are all synonymous. When any of the chemical groups or compound classes defined herein is modified by the term “substituted”, any carbon atom(s) in the moiety replacing a hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic R system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ^{t}Bu), and —CH₂C(CH₃)₃(neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The term “alkyl” includes straight or branched hydrocarbon groups having 1-10 carbon atoms and includes, for example, methyl, ethyl, n-propyl, isopropyl, n-butyl, sec-butyl, isobutyl, tert-butyl, n-pentyl, n-hexyl, n-heptyl, n-octyl, n-nonyl, n-decyl, fluoromethyl, fluorochloromethyl, and trifluoromethyl, and the like. Alkyl groups may be optionally substituted with one or more substituents, such as halogens. The term “branched” should be understood to represent a linear straight chain hydrocarbon group having one or more lower alkyl groups such as methyl, ethyl or propyl, attached to it. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e., —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

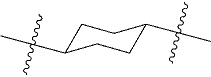 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” refers to an alkyl group containing at least one carbon-carbon double bond. Alkenyl groups may be optionally substituted with one or more substituents. The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” refers to an alkyl group containing at least one carbon-carbon triple bond. Alkynyl groups may be optionally substituted with one or more substituents. The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen.

As used herein, the term alkynyl does not preclude the presence of one or more non-aromatic carbon-carbon double bonds. The groups —C—CH, —C—CCH₃, and —CH₂C—CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structure, wherein the ring atoms are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl or cycloalkyl groups (carbon number limitation permitting) attached to the first aromatic ring or any additional aromatic ring present. If a cycloalkyl groups is present, such a group may be fused to one or more of the aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

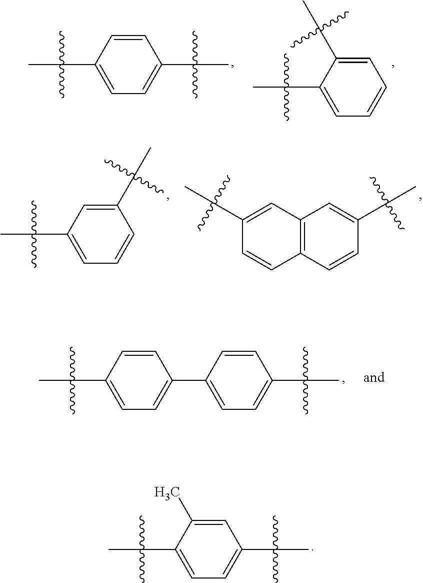

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, the aromatic ring structures being one, two, three, or four ring structures each containing from three to nine ring atoms, and wherein the heteroaryl group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroaryl does not preclude the presence of one or more alkyl or aryl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl.

The term “heteroaryl” includes aromatic mono- or bicyclic rings incorporating one or more (e.g., 1-4) heteroatoms selected from N, O, and S. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused 5- and 6-membered rings or two fused 6-membered rings. Each ring may contain up to about four heteroatoms typically selected from N, O, and S. Typically the heteroaryl ring will contain up to 3 heteroatoms, more usually up to 2, for example a single heteroatom. In one embodiment, the heteroaryl ring contains at least one ring N atom. The N atoms in the heteroaryl rings can be basic, as in the case of an imidazole or pyridine, or essentially non-basic as in the case of an indole or pyrrole nitrogen. In general, the number of basic N atoms present in the heteroaryl group, including any amino group substituents of the ring, will be less than 5. Examples of heteroaryl include, but are not limited to, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, and imidazo[1,2-b][1,2,4]triazinyl. Examples of heteroaryl groups comprising at least one N in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl. “Heteroaryl” also covers partially aromatic bi- or polycyclic ring systems wherein at least one ring is an aromatic ring and one or more of the other rings is a non-aromatic, saturated or partially saturated ring, provided at least one ring contains one or more heteroatoms selected from N, O, and S. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, and 3,4-dihydro-2H-pyrido[3,2-b][1,4]oxazinyl.

The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroarenediyl does not preclude the presence of one or more alkyl or aryl groups (carbon number limitation permitting) attached to the aromatic ring or aromatic ring system. Non-limiting examples of heteroarenediyl groups include:

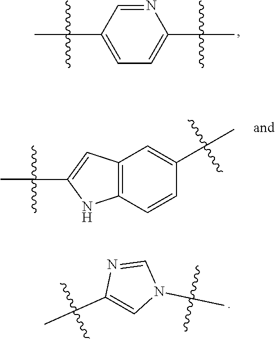 [SMILES: *c1ccc(*)nc1 ; *c1ccc2[nH]c(*)cc2c1 ; *c1cn(*)cn1]

The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, the non-aromatic ring structures being one, two, three, or four ring structures each containing from three to nine ring atoms, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic.

Non-limiting examples of heterocycloalkyl groups include cyclic ethers such as oxiranyl, oxetanyl, tetrahydrofuranyl, dioxanyl, and substituted cyclic ethers. Heterocycloalkyl rings comprising at least one N in a ring position include, for example, aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, tetrahydrotriazinyl, tetrahydropyrazolyl, tetrahydropyridinyl, homopiperidinyl, homopiperazinyl, 3,8-diaza-bicyclo[3.2.1]octanyl, 8-aza-bicyclo[3.2.1]octanyl, 2,5-Diaza-bicyclo[2.2.1]heptanyl and the like. Typical sulfur containing heterocycloalkyl rings include tetrahydrothienyl, dihydro-1,3-dithiol, tetrahydro-2H-thiopyran, and hexahydrothiepine. Other heterocycloalkyl rings include oxiranyl, oxetanyl, dihydrooxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolanyl, octahydrobenzofuranyl, octahydrobenzimidazolyl, and octahydrobenzothiazolyl. For heterocycles containing S, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. 1 and 2 oxo (═O) heterocyclyl groups include for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from N, O, or S, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, the term “piperidino” or “morpholino” refers to a piperidin-1-yl or morpholin-4-yl ring that is linked via the ring nitrogen. Non-limiting examples of heterocycloalkyl groups include the term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings are connected with a covalent bond. As used herein, the term heterocycloalkanediyl does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

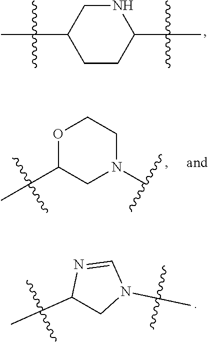 [SMILES: *C1CCC(*)NC1 ; *C1CN(*)CCO1 ; *C1CN(*)C=N1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), or —OC(CH₃)₃(tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively.

The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The term “hydroxypropyl” refers to three-carbon groups comprising one hydroxyl group and includes, but is not limited to, 2-hydroxypropyl and 1-hydroxypropan-2-yl. The term “dihydroxypropyl” refers to three-carbon groups comprising two hydroxyl groups and includes, but is not limited to, 1,3-dihydroxypropan-2-yl and 2,3-dihydroxypropyl.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The terms ortho, meta and para substitution are well understood in the art. For the absence of doubt, “ortho” substitution is a substitution pattern where adjacent carbons possess a substituent. “Meta” substitution is a substitution pattern where two substituents are on carbons one carbon removed from each other, i.e., with a single carbon atom between the substituted carbons. In other words there is a substituent on the second atom away from the atom with another substituent. “Para” substitution is a substitution pattern where two substituents are on carbons two carbons removed from each other, i.e., with two carbon atoms between the substituted carbons. That is, there is a substituent on the third atom away from the atom with another substituent.

Where used herein, the term “weak base” refers to compounds that accept protons weakly. Examples include but are nor limited to ammonia and sodium bicarbonate.

Where used herein, the term “weak acid” refers to compounds that have a weak tendency to donate protons. Examples include but are not limited to acetic acid, and citric acid.

Where used herein, the term “strong base” refers to compounds that readily accepts protons, and the term “strong acid” refers to compounds that have a strong tendency to donate protons.

The following standard amino acid abbreviations may be used herein: Alanine:ala:A, Cysteine:cys:C, Aspartic acid:asp:D, Glutamic acid:glu:E, Phenylalanine:phe:F, Glycine:gly:G, Histidine:his:H, Isoleucine:ile:I, Lysine:lys:K, Leucine:leu:L, Methionine:met:M, Asparagine:asn:N, Glutamine:gln:Q, Proline:pro:P, Arginine:arg:R, Serine:ser:S, Threonine:thr:T, Valine:val:V, Tryptophan:trp:W, and Tyrosine:tyr:T.

Particular examples of conservative amino acid substitutions include, but are not limited to, gly:ala substitutions; val:ile:leu substitutions; asn:glu:his substitutions; asp:glu substitutions; ser:thr:met substitutions; lys:arg:his substitutions; and phe:tyr:trp substitutions. Other types of substitutions, variations, additions, deletions and derivatives that result in functional variant peptides are also encompassed by the present disclosure, and one of skill in the art would readily know how to make, identify, or select such variants or derivatives, and how to test for receptor binding activity of those variants. Examples of conservative amino acid substitutions include, but are not limited to, substitutions made within the same group such as within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, and valine), aromatic amino acids (such as phenylalanine, tryptophan, tyrosine) and small amino acids (such as glycine, alanine, serine, threonine, methionine). Other examples of possible substitutions are described below.

Peptides of the present disclosure and the nucleic acids which encode them include peptide and nucleic acid variants which comprise substitutions (conservative or non-conservative) of the native amino acids or bases. For example, the peptide variants include, but are not limited to, variants that are not exactly the same as the sequences disclosed herein, but which have, in addition to the substitutions explicitly described for various sequences listed herein, additional substitutions of amino acid residues (conservative or non-conservative) which substantially do not impair the activity or properties of the variants described herein. Examples of such conservative amino acid substitutions may include, but are not limited to, ala to gly, ser, or thr; arg to gln, his, or lys; asn to asp, gln, his, lys, ser, or thr; asp to asn or glu; cys to ser; gln to arg, asn, glu, his, lys, or met; glu to asp, gln, or lys; gly to pro or ala; his to arg, asn, gln, or tyr; ile to leu, met, or val; leu to ile, met, phe, or val; lys to arg, asn, gln, or glu; met to gln, ile, leu, or val; phe to leu, met, trp, or tyr; ser to ala, asn, met, or thr; thr to ala, asn, ser, or met; trp to phe or tyr; tyr to his, phe or trp; and val to ile, leu, or met.

The terms “peptide,” “peptide analog,” “peptide derivative,” or “peptide compound,” where used herein may refer to a molecule comprising only amino acids, or may refer to a molecule comprising amino acids and one or more non-amino acid structures (e.g., poly(ethylene glycol) (PEG) units), and may refer to a variant (“mutant”) of a “wild-type” peptide, or to a molecule comprising amino acids and one or more non-amino acid structures (e.g., PEG units).

The term “mutant” or “variant” is intended to refer to a protein, peptide, nucleic acid or organism which has at least one amino acid or nucleotide which is different from the wild-type version of the protein, peptide, nucleic acid, or organism and includes, but is not limited to, point substitutions, multiple contiguous or non-contiguous substitutions, insertions, chimeras, or fusion proteins, and the nucleic acids which encode them, or other non-wild-type features as described herein.

The terms “peptide” or “peptide sequence” are used herein to designate a series of amino acid residues, connected one to another. In natural (“wild-type”) peptides, amino acids are connected by peptide bonds between the alpha-amino and carbonyl groups of the adjacent amino acids to form an amino acid sequence. In certain embodiments, the peptides can range in length from 5 to 15 to 25 to 40 to 60 to 75 amino acids or more, for example, 5, 6, 7, 8, 9, 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 to 75 to 100 amino acids, or more. The term “polypeptide” or “protein” is used herein to designate a series of amino acid residues, connected one to the other typically by peptide bonds between the alpha-amino and carbonyl groups of the adjacent amino acids, wherein the length is longer than a single peptide. A “fusion protein” or “fusion polypeptide” refers to proteins or polypeptides (and may be used interchangeably) which have been created by recombinant or synthetic methods to combine peptides in a serial configuration.

The term “polypeptide” or “protein” is used herein to designate a series of amino acid residues, connected one to the other typically by peptide bonds between the alpha-amino and carbonyl groups of the adjacent amino acids, wherein the length is longer than a single peptide, or by other connecting bonds as described herein. A peptide compound of the present disclosure may be a peptide conjugate, which in a non-limiting embodiment is a compound comprising a peptide of the present disclosure which is conjugated (e.g., covalently linked, directly or indirectly via a linker sequence) to another molecule, such as (but not limited to) a carrier molecule such as (but not limited to) a protein or other polymeric molecule, e.g., a serum albumin molecule or antibody, or other therapeutic compound such as (but not limited to) a drug, or an imaging or diagnostic moiety, and wherein the peptide retains its activity (e.g., binding, targeting, imaging, or inhibitory) even when conjugated to the molecule. The peptides of the present disclosure may be produced using any nucleotide sequence which encodes the desired amino acid sequence. Any of the peptides described herein or active variants thereof may be used to make the peptide conjugates of the present disclosure.

Where used herein, the term “wild-type” refers to an amino acid sequence or peptide which occurs under natural conditions or in nature, as opposed to a “non-wild-type” amino acid sequence or peptide which does not occur under natural conditions or in nature. A non-wild-type amino acid sequence or peptide may be an amino acid sequence or peptide that differs from the wild-type such as via substitution, deletion, or insertion of one or more natural (alpha) amino acids in one or more amino acid positions of the wild-type amino acid sequence or peptide. A non-wild-type amino acid sequence or peptide may also be an amino acid sequence or peptide that differs from the wild-type by one or more substitutions with a corresponding D-amino acid, R amino acid, homo-amino acid, R-homo amino acid, or peptoid monomer analog thereof. For example, where the wild-type peptide comprises a serine, the non-wild-type may instead comprise a D-serine, R serine, homoserine, 0-homoserine, or peptoid monomer analog of serine. The non-wild-type may differ from the wild-type in only one amino acid position, or in a subset of the amino acid positions, or in all of the amino acid positions. For example, a peptide which comprises only D-amino acids is known as a retro-inverso peptide. A non-wild-type amino acid or peptide may also be one in which the two or more of the amino acid monomers are linked via a non-peptide bond, such as a peptoid bond. In certain embodiments the non-peptide bond of the non-wild-type may be of the following types: reverse peptide bond (—NH—CO—), —CH₂—NH—, —CH₂S—, —CH₂CH₂—, —CH═CH—, —COCH₂—, —CH(OH)CH₂—, and —CH₂SO—. Exemplary non-peptide bonds which may be employed are described in U.S. Pat. Nos. 4,897,445 and 10,064,926 for example. A peptide compound of the present disclosure may also comprise a wild-type amino acid sequence or peptide which is conjugated to another natural peptide or amino acid sequence, which when conjugated together comprise a synthetic or non-natural amino acid sequence or peptide. A peptide compound of the present disclosure may also comprise a wild-type amino acid sequence or peptide which is conjugated to a non-natural amino acid sequence or peptide, e.g., one or more D-amino acids, β amino acids, homo-amino acids, R-homo amino acids, or peptoid monomer analogs. A peptide compound of the present disclosure may comprise a wild-type amino acid sequence or peptide conjugated to a non-amino acid molecule, such as a PEG molecule, for enhancing solubility, penetrability, or resistance to enzymatic degradation. In another embodiment, a peptide may be “stapled” into a particular helical configuration. Such “stapling” within peptides is also considered to be non-wild type difference from wild-type peptides. Stapled peptides are discussed in further detail below.

The terms “synthetic amino acid” and “non-natural amino acid” may be used in place of the term non-wild-type, and also refer to an organic compound that has a structure similar to a natural amino acid so that it mimics the structure and reactivity of a natural amino acid. The synthetic amino acid as defined herein generally increases or enhances the properties of a peptide (e.g., selectivity, stability) when the synthetic amino acid is either substituted for a natural amino acid or incorporated into a peptide.

A non-wild-type homolog of a wild-type amino acid sequence or peptide therefore refers to a non-wild-type amino acid sequence or peptide which has at least one difference from the wild-type amino acid sequence or peptide in at least one way as set forth above.

The term non-wild-type, when used in reference to a single amino acid molecule, may also refer to a single amino acid or amino acid analog such as a peptoid monomer, which does not occur under natural conditions or in nature, as opposed to a wild-type amino acid.

Non-wild-type amino acid sequences and peptides of the present disclosure may include the common natural amino acids alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine as well as less common naturally occurring amino acids, modified amino acids or synthetic compounds, including but not limited to: alpha-asparagine, 2-aminobutanoic acid, 2-aminobutyric acid, 4-aminobutyric acid, 2-aminocapric acid (2-aminodecanoic acid), 6-aminocaproic acid, alpha-glutamine, 2-aminoheptanoic acid, 6-aminohexanoic acid, alpha-aminoisobutyric acid (2-aminoalanine), 3-aminoisobutyric acid, beta-alanine, allo-hydroxylysine, allo-sioleucine, 4-amino-7-methylheptanoic acid, 4-amino-5-phenylpentanoic acid, 2-aminopimelic acid, gamma-amino-beta-hydroxybenzenepentanoic acid, 2-aminosuberic acid, 2-carboxyazetidine, beta-alanine, beta-aspartic acid, biphenylalanine, 3,6-diaminohexanoic acid, butanoic acid, cyclobutyl alanine, cyclohexylalanine, cyclohexylglycine, N5-aminocarbonylornithine, cyclopentyl alanine, cyclopropyl alanine, 3-sulfoalanine, 2,4-diaminobutanoic acid, diaminopropionic acid, 2,4-diaminobutyric acid, diphenyl alanine, NN-dimethylglycine, diaminopimelic acid, 2,3-diaminopropanoic acid, S-ethylthiocysteine, N-ethylasparagine, N-ethylglycine, 4-aza-phenylalanine, 4-fluoro-phenylalanine, gamma-glutamic acid, gamma-carboxyglutamic acid, hydroxyacetic acid, pyroglutamic acid, homoarginine, homocysteic acid, homocysteine, homohistidine, 2-hydroxyisovaleric acid, homophenylalanine, homoleucine, homoproline, homoserine, homoserine, 2-hydroxypentanoic acid, 5-hydroxylysine, 4-hydroxyproline, 2-carboxyoctahydroindole, 3-carboxyisoquinoline, isovaline, 2-hydroxypropanoic acid (lactic acid), mercaptoacetic acid, mercaptobutanoic acid, sarcosine, 4-methyl-3-hydroxyproline, mercaptopropanoic acid, norleucine, nipecotic acid, nortyrosine, norvaline, omega-amino acid, ornithine, penicillamine (3-mercaptovaline), 2-phenylglycine, 2-carboxypiperidine, sarcosine (N-methylglycine), 2-amino-3-(4-sulfophenyl)propionic acid, 1-amino-1-carboxycyclopentane, 3-thienylalanine, epsilon-N-trimethyllysine, 3-thiazolylalanine, thiazolidine 4-carboxylic acid, alpha-amino-2,4-dioxopyrimidinepropanoic acid, and 2-naphthylalanine.

The term amine as used herein may refer, for example, to alkyl amines such as methyl amine, ethyl amine, dimethyl amine, diethyl amine, trimethyl amine, triethyl amine, diethanolamine, triethanolamine, and/or trimethylammonia, and combinations thereof. The term amine may further refer to an acyclic or cyclic polyamine such as, for example, spermine, spermidine, tris(2-aminoethyl)amine, cyclen, cyclam, 1,4,7-triazacyclononane, 1,1,1-tris(aminomethyl)ethane, ethylenediamine, 1,4-diazabicyclo[2.2.2]octane (DABCO), diethylenetriamine, triethylenetetramine, 1,3-diaminopropane, putrescine, cadaverine, sym-norspermidine, sym-homospermidine, norspermine, thermospermine, carboxyspermidine, norcarboxyspermidine, caldopentamine, caldohexamine, ethylenediamine, 1,2-diaminopropane, 1,3-diaminopropane, N-methylethylenediamine, 1,4-diaminobutane, 3-(methylamino)propylamine, N,N′-dimethylethylenediamine, N-methyl-1,3-diaminopropane, 1-dimethylamino-2-propylamine, 3-(dimethylamino)-1-propylamine, N,N,N′,N′-tetramethyldiaminomethane, N,N,N′-trimethylethylenediamine, N-isopropylethylenediamine, N-propylethylenediamine, 2-(aminomethyl)-2-methyl-1,3-propanediamine, 1,2-diamino-5-bromo-3-chlorobenzene, 3,5-dichloro-1,2-diaminobenzene, 4-bromo-1,2-diaminobenzene, 4,5-dichloro-o-phenylenediamine, 4-chloro-1,3-diaminobenzene, 2-nitro-1,4-phenylenediamine, 3-nitro-1,2-phenylenediamine, 4-nitro-o-phenylenediamine, m-phenylenediamine, o-phenylenediamine, p-phenylenediamine, trans-4-cyclohexene-1,2-diamine, cis-4-cyclohexene-1,2-diamine, hexamethylenetetramine, 4-aminobenzylamine, N,N′-bis(2-aminoethyl)-1,3-propanediamine, methyl 3,4-diaminobenzoate, 1,2-diamino-3,5-dimethylbenzene, 4,5-dimethyl-1,2-phenylenediamine, 4-(2-aminoethyl)aniline, aniline, m-xylylenediamine, N-phenylethylenediamine, o-xylylenediamine, p-xylylenediamine, 1,8-diaminooctane, N,N-dimethyldipropylenetriamine, 1,2-bis(3-aminopropylamino)ethane, N-tosylethylenediamine, 2,2,4(2,4,4)-trimethyl-1,6-hexanediamine, 1,4-diaminonaphthalene, 1,5-diaminonaphthalene, 1,8-diaminonaphthalene, 4-tert-butyl-2,6-diaminoanisole, 2,2′-oxydianiline, 4,4′-oxydianiline, 3,3′-diaminobenzidine, 3,4′-diaminodiphenylmethane, 4,4′-diaminodiphenylmethane, 4,4′-ethylenedianiline, 2,4,6-triethyl-1,3,5-benzenetrimethanamine, and/or 1,8-anthracenedimethanamine.

The term “nucleic acid” is well known in the art and as used herein generally refers to a molecule (i.e., a strand) of deoxyribonucleic acid (DNA), ribonucleic acid (RNA), or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an “A,” a “G,” a uracil “U” or a “C”). The term nucleobase also includes non-natural bases as described below. The term “nucleic acid” encompasses the terms “oligonucleotide” and “polynucleotide,” each as a subgenus of the term “nucleic acid.” The term “oligonucleotide” generally refers to a molecule of between about 3 and about 100 nucleobases in length. The term “polynucleotide” generally refers to at least one molecule of greater than about 100 nucleobases in length. These definitions generally refer to a single-stranded molecule, but in specific embodiments will also encompass an additional strand that is partially, substantially or fully complementary to the single-stranded molecule. Thus, a nucleic acid may encompass a double-stranded molecule that comprises a complementary strand or “complement” of a particular sequence comprising a molecule. As used herein, a single-stranded nucleic acid may be denoted by the prefix “ss,” and a double-stranded nucleic acid by the prefix “ds.” The terms “polynucleotide sequence” or “nucleic acid,” as used herein, include any polynucleotide sequence which encodes a peptide or fusion protein (or polypeptide) including polynucleotides in the form of RNA, such as mRNA, or in the form of DNA, including, for instance, cDNA and genomic DNA obtained by cloning or produced by chemical synthetic techniques or by a combination thereof. The RNA or DNA may be double-stranded or single-stranded. Single-stranded DNA may be the coding strand, also known as the sense strand, or it may be the non-coding strand, also referred to as the anti-sense strand.

Where a strand is designated herein as RNA, and thus comprises uracil (U) nucleobases, the present disclosure is also directed to an equivalent DNA sequence where the U nucleobase is replaced with a thymine (T) nucleobase. For example, where an RNA active agent described herein comprises the seed sequence GUCUGA, the equivalent DNA active agent comprises the seed sequence GTCTGA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Therefore, in the context of the present disclosure, the term “oligonucleotide” refers to an oligomer or polymer of RNA or DNA or mimetics thereof. This term includes oligonucleotides composed of naturally-occurring nucleobases, sugars and covalent internucleoside (backbone) linkages as well as oligonucleotides having non-naturally-occurring nucleobases, sugars and synthetic heterocycles and covalent internucleoside (backbone) linkages which function similarly. Such modified or substituted non-natural oligonucleotides, as compared to native (natural) forms may have desirable properties such as, for example, enhanced cellular uptake, enhanced affinity for nucleic acid target and increased stability in the presence of nucleases.

Where used herein, the term “oligonucleotide,” is also intended to include linked nucleobase sequences containing modified backbones comprising non-natural internucleoside linkages. As defined in this specification, oligonucleotides having modified backbones include those that retain a phosphorus atom in the backbone and those that do not have a phosphorus atom in the backbone. Further, for the purposes of this specification, the term “nucleoside” is intended to refer to a nucleobase linked to a ribose or deoxyribose sugar (a natural nucleoside), and to a nucleobase linked to a non-ribose or non-deoxyribose heterocycle, e.g., a morpholine structure (a non-natural, or modified, nucleoside or other structures described elsewhere herein). Thus, a series of such modified, non-natural, nucleosides linked together via an internucleoside backbone can also be considered to be an oligonucleotide (a non-natural, or modified, oligonucleotide). Further, the term “sugar,” where used herein in the context of a nucleoside, is intended to include “non-sugar” heterocyclic compounds, such as morpholines, as the portion of the internucleoside backbone which is linked to the nucleobase.

Oligonucleotides useful in the compounds and methods disclosed herein also include those comprising entirely or partially of naturally occurring nucleobases. Naturally occurring nucleobases as defined herein, include adenine, guanine, thymine, cytosine, and uracil. Although 5-methylcytosine (5-me-C) is technically a naturally occurring nucleobase, for the purposes of the present disclosure it will be included in the list of non-natural (a.k.a., modified) nucleobases.

As noted above, oligonucleotides of the present disclosure may further include those comprised entirely or partially of modified nucleobases and their corresponding nucleosides. These modified nucleobases include, but are not limited to, 5-uracil (pseudouridine), dihydrouracil, inosine, ribothymine, 5-me-C, 7-methylguanine, hypoxanthine, xanthine, 5-hydroxymethyl cytosine, 2-aminoadenine, 2-methyladenine, 6-methyladenine, 2-propyladenine, N6-adenine, N6-isopentenyladenine, 2-methylthio-N6-isopentenyladenine, 2-methylguanine, 6-methylguanine, 2-propylguanine, 1-methylguanine, 7-methylguanine, 2,2-dimethylguanine, 2-thiouracil, 2-thiothymine, 2-thiocytosine, 5-fluorouracil, 5-bromouracil, 5-chlorouracil, 5-iodouracil, dihydrouracil, 5-methyl-2-thiouracil, 2-thiouracil, 4-thiouracil, 5-methyluracil, uracil-5-oxyacetic acid methylester, uracil-5-oxyacetic acid, 5-carboxymethylaminomethyl-2-thiouracil, hypoxanthine, xanthine, 4-acetylcytosine, 5-(carboxyhydroxylmethyl) uracil, 5-methoxycarboxymethyluracil, 5-methoxyuracil, 5-methyl-2-thiouracil, 3-(3-amino-3-N-2-carboxypropyl) uracil, 5-carboxymethylaminomethyluracil, 5-methylaminomethyluracil, 5-methoxyaminomethyl-2-thiouracil, alkynyl derivatives of pyrimidine bases including 5-propynyl uracil, and 5-propynyl cytosine, 6-azo uracil, 6-azo cytosine, 6-azo thymine, 4-thiouracil, 8-halo-adenines, 8-amino adenine, 8-thiol adenine, 8-thioalkyl adenine, 8-hydroxyl adenine, 5-trifluoromethyl uracil, 3-methylcytosine, 5-methylcytosine, 5-trifluoromethylcytosine, 7-methylguanine, 7-methyladenine, 2-F-adenine, 2-amino-adenine, 8-azaguanine and 8-azaadenine, 7-deazaguanine, 8-halo-guanines, 8-amino guanine, 8-thiol guanine, 8-thioalkyl guanine, 8-hydroxyl guanine, 7-deazaadenine, 3-deazaguanine, 3-deazaadenine, beta-D-galactosylqueosine, beta-D-mannosylqueosine, 1-methylinosine, 2,6-diaminopurine, queosine, tricyclic pyrimidines, phenoxazine cytidine(1H-pyrimido[5,4-b][1,4]benzoxazin-2(3H)-one), and phenothiazine cytidine (1H-pyrimido[5,4-b][1,4]benzothiazin-2(3H)-one.

The present disclosure also encompasses oligonucleotides which comprise targeting sequences (base sequences) that are complementary to particular nucleic acid target sequences taught herein. A nucleic acid is a “complement” or is “complementary” to another nucleic acid when it is capable of base-pairing with the other nucleic acid according to the standard Watson-Crick, Hoogsteen or reverse Hoogsteen binding complementarity rules. Polynucleotides (nucleic acids) are described as “complementary” to one another when hybridization occurs in an antiparallel configuration between two single-stranded polynucleotides.

More particularly, “complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target, and as such, as is understood in the art, the targeting sequence of an antisense oligonucleotide of the present disclosure need not be 100% complementary to that of its target sequence to be specifically hybridizable. An oligonucleotide is specifically hybridizable when binding of the oligonucleotide to the target sequence of the DNA or RNA molecule interferes with the normal function of the target DNA or RNA to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment, and in the case of in vitro assays, under conditions in which the assays are performed. An oligonucleotide and a target sequence are thus complementary to each other when a sufficient number of nucleobases of the oligonucleotide can hydrogen bond with the corresponding nucleobases of the target sequence, such that a desired effect will occur (e.g., antisense inhibition of a target nucleic acid, such as an AR coregulator).

For example, an oligonucleotide in which 18 of 20 nucleobases of the oligonucleotide are complementary to a target sequence, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligonucleotide which is 18 nucleobases in length having three noncomplementary nucleobases which are flanked by two regions of complete complementarity with the target nucleic acid, or are distributed in non-contiguous positions, would have 83% overall complementarity with the target sequence.

Where used herein, in at least certain embodiments, the term “carbohydrate” may be used to refer to alcohols, alditols, glycols, polyols, monosaccharides, disaccharides (two monosaccharides linked together), oligosaccharides (three or more (e.g., 3-10) monosaccharides liked together), polysaccharides (polymers comprising ten or more linked monosaccharides), and glycosylamines (amino sugars). The alcohols, alditols, glycols, polyols, monosaccharides (e.g., pentoses and hexoses), disaccharides, oligosaccharides, and/or polysaccharides may be, for example, cyclitol, acarviocin, aminocyclitol, bornesitol, ciceritol, conduritol, decahydroxycyclopentane, 5-deoxyinositol, dodecahydroxycyclohexane, ononitol, pinitol, pinpollitol, quebrachitol, theogallin, 3,4,5-tri-O-galloylquinic acid, inositol, inositol pentakisphosphate, cis-inositol, D-chiro-inositol, L-chiro-inositol, epi-inositol, neo-inositol, muco-inositol, neo-inositol, scyllo-inositol, sorbitol, threitol, arabitol, galactitol, iditol, volemitol, sorbitol, fucitol, xylitol, lactitol, erythritol, lactitol, maltitol, phytic acid, quinic acid, 2-methoxyethan-1-ol (methyl glycol), propylene glycol, 1,2-propanediol, ethylene glycol, low molecular weight polyethylene glycols (e.g., C2-C10), vegetable glycerine, dipropylene glycol, glycerol, panthenol, cytosine glycol, cyclohexane-1,2-diol, aminomethanol, ethyleneglycol, 1,3-propanediol, 1,4-butanediol, 2,2-dimethyl-1-butanol, ethanol, propanol, butanol, pentanol, hexanol, ethynol, acetylenediol, fenticlor, fucitol, gluconic acid, glucic acid, 2-heptanol, 3-heptanol, 2-hexanol, 3-hexanol, ribitol, ethylhexylglycerin, octoxyglycerin, glucuronic acid, glyceraldehyde, glyceric acid, glycerol 3-phosphate, glycerol monostearate, 2-octanediol, pinacol, racemic acid, tartaric acid, uronic acid, xylosan, 1,2-butanediol, 1,3-butanediol, 1,4-butanediol, 2,3-butanediol, 2-methyl-2,4-pentanediol, neopentyl glycol, erythulose, arabinose, bis-HPPP, cellobiose, mannitol, mannose, glucose, ribose, allose, altrose, gulose, idose, lactose, maltose, dextrose, galactose, talose, psicose, fructose, sorbose, tagatose, β-d-ribopyranose, u-d-ribopyranose, β-d-ribofuranose, u-d-ribofuranose, sucrose, xylose, trehalose, raffinose, stachyose, sucralose, isomalt, isomaltulose, maltodextrins, fructo-oligosaccharides, amylose, amylopectin, starch, glycogen, cellulose, hemicellulose, methyl cellulose, methyl ethyl cellulose, pectins, hydrocolloids, isomaltooligosaccharide, maltodextrin, and/or polydextrose, glucosamine, galactosamine, N-acetyl glucosamine, N-acetyl galactosamine, mannosamine, neuraminic acid, bacillosamine, and combinations thereof.

The terms “subject” and “patient” are used interchangeably herein and will be understood to refer to a warm-blooded animal, particularly a mammal. Non-limiting examples of animals within the scope and meaning of this term include dogs, cats, rabbits, rats, mice, guinea pigs, chinchillas, hamsters, ferrets, horses, pigs, goats, cattle, sheep, zoo animals, camels, llamas, non-human primates, including Old and New World monkeys and non-human primates (e.g., cynomolgus macaques, chimpanzees, rhesus monkeys, orangutans, and baboons), and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic effect without excessive adverse side effects (such as toxicity, irritation and allergic response) commensurate with a reasonable benefit/risk ratio when used in the manner of the inventive concepts. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

When more than one of the active agents described in present disclosure, or their equivalents, are administered, they may be used or administered conjointly. As used herein the terms “conjointly” or “conjoint administration” refers to any form of administration of two or more different biologically-active compounds (i.e., active agents) such that the second compound is administered while the previously administered therapeutic compound is still effective in the body, whereby the two or more compounds are simultaneously active in the patient. For example, the different therapeutic compounds can be administered either in the same formulation, or in separate formulations, either concomitantly (together) or sequentially. When administered sequentially the different compounds may be administered immediately in succession, or separated by a suitable duration of time, as long as the active agents function together in a synergistic manner. In certain embodiments, the different therapeutic compounds can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus an individual who receives such treatment can benefit from a combined effect of the different therapeutic compounds.

The DMX-5804 and analogs and derivatives thereof which are disclosed herein, such as glycoconjugates thereof, may be linked to carrier molecules for enhancing delivery of the DMX-5804 and analogs and derivatives thereof to target specific cells and tissues which express MAP4K4, such as but not limited to cardiomyocytes, neurons, skeletal muscle cells, colorectal cancer cells, hepatocellular carcinoma cells, pancreatic ductal adenocarcinoma cells, lung adenocarcinoma cells, prostate cancer cells, and cells of cancers in which inhibition of tumor cell motility is desired.

In at least certain embodiments, the active agents of the present disclosure (including but not limited to, DMX-5804 and analogs and derivatives thereof as described elsewhere herein, may be combined with a pharmaceutically acceptable component (e.g., a carrier, vehicle, excipient, and/or diluent) to form a pharmaceutical composition for use in accordance with the methods of the present disclosure, for example for treating cancer, stroke, and myocardial infarction (MI). Such a composition may contain, in addition to the active agent and carrier, diluents, fillers, salts, buffers, stabilizers, solubilizers, and other materials well known in the art. Suitable carriers, vehicles and other components of the formulation are described, for example, in Remington: The Science and Practice of Pharmacy, 22^{nd} ed. The term “pharmaceutically acceptable” means a non-toxic material that does not interfere with the effectiveness of the biological activity of the active agent. The characteristics of the carrier will depend on the route of administration.

For example, but not by way of limitation, the active agent may be dissolved, suspended, or emulsified in a physiologically acceptable pharmaceutical carrier or diluent and administered as either a solution or a suspension. Non-limiting examples of suitable pharmaceutically acceptable carriers include water, saline, dextrose solutions, fructose solutions, ethanol, or oils of animal, vegetative, or synthetic origin, or any combination thereof. A sterile diluent, which may contain materials generally recognized for approximating physiological conditions and/or as required by governmental regulations, may be employed as the pharmaceutically acceptable carrier. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use.

The pharmaceutical compositions may also contain one or more additional components in addition to the active agent and pharmaceutically acceptable carrier(s) (and other additional therapeutically active agent(s), if present). Examples of additional components that may be present include, but are not limited to, diluents, fillers, salts, buffers, preservatives, stabilizers, solubilizers, and other materials well known in the art. Another particular non-limiting example of an additional component that may be present in the pharmaceutical composition is a delivery agent, as discussed in further detail herein below.

Some examples of suitable excipients or carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, poly(ethyleneglycol), cellulose, sterile water, syrup, and methyl cellulose. The formulations can additionally include: lubricating agents such as (but not limited to) talc, magnesium stearate, and mineral oil; wetting agents; emulsifying and suspending agents; preserving agents such as (but not limited to) methyl- and propylhydroxy-benzoates; sweetening agents; and flavoring agents. The compositions can be formulated so as to provide quick, sustained, or delayed release of the active ingredient after administration to the patient by employing procedures known in the art.

For preparing solid compositions such as (but not limited to) tablets or other solid dosage forms, the principal active ingredient can be mixed with a pharmaceutical excipient or carrier to form a solid preformulation composition containing a homogeneous mixture of the active agent. When referring to these preformulation compositions as homogeneous, it is meant that the active agent is dispersed evenly throughout the composition so that the composition may be readily subdivided into equally effective unit dosage forms such as tablets, pills, and capsules.

The dosage forms may be coated or otherwise compounded to provide a dosage form affording the advantage of prolonged action. For example, the tablet or pill can comprise an inner dosage component and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present disclosure may be incorporated for administration orally or by injection include (but are not limited to) aqueous solutions, suitably flavored syrups, aqueous or oil suspensions, and flavored emulsions with edible oils such as (but not limited to) corn oil, cottonseed oil, sesame oil, coconut oil, or peanut oil, as well as elixirs and similar pharmaceutical vehicles. In some embodiments, the active agent is administered in solution. The formulation thereof may be in a solution having a suitable pharmaceutically acceptable buffer such as (but not limited to) phosphate, Tris (hydroxymethyl) aminomethane-HCl or citrate, and the like. Buffer concentrations should be in the range of 1 to 100 mM. The formulated solution may also contain a salt, such as (but not limited to) sodium chloride or potassium chloride, in a concentration of 50 mM to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

Other embodiments of the pharmaceutical compositions of the present disclosure may include the incorporation or entrapment of the active agent in various types of drug delivery systems that function to provide targeted delivery, controlled release, and/or increased half-life to the active agent. For example, but not by way of limitation, it is possible to entrap the active agent in microcapsules prepared by coacervation techniques or by interfacial polymerization (for example, hydroxymethylcellulose or gelatin-microcapsules and poly-(methylmethacylate) microcapsules, respectively). It is also possible to entrap the active agent in macroemulsions or colloidal drug delivery systems (such as but not limited to, liposomes, albumin microspheres, microemulsions, nanoparticles, nanocapsules, and the like). Such techniques are well known to persons having ordinary skill in the art, and thus no further description thereof is deemed necessary.

Compositions for inhalation or insufflation include solutions and suspensions in pharmaceutically acceptable, aqueous, or organic solvents, or mixtures thereof, and powders. The liquid or solid compositions may contain suitable pharmaceutically acceptable excipients as described supra. The compositions can be administered by the oral or nasal respiratory route for local or systemic effect. Compositions in pharmaceutically acceptable solvents may be nebulized by use of inert gases. Nebulized solutions may be inhaled directly from the nebulizing device, or the nebulizing device may be attached to a face mask tent or intermittent positive pressure breathing machine. Solution, suspension, or powder compositions may also be administered orally or nasally from devices which deliver the formulation in an appropriate manner.

As noted, the active agent can be combined with a pharmaceutically acceptable carrier (excipient) or vehicle to form a pharmacological composition. Pharmaceutically acceptable carriers can contain a physiologically acceptable compound that acts to, e.g., stabilize, or increase or decrease the absorption or clearance rates of the pharmaceutical compositions. Physiologically acceptable carriers and vehicles can include, for example, carbohydrates, such as glucose, sucrose, or dextrans, antioxidants, such as ascorbic acid or glutathione, chelating agents, low molecular weight proteins, detergents, liposomal carriers, or excipients or other stabilizers and/or buffers. Other physiologically acceptable compounds, carriers, and vehicles include wetting agents, emulsifying agents, dispersing agents or preservatives.

When administered orally, the present compositions may be protected from digestion. This can be accomplished either by complexing the active agent with a composition to render it resistant to acidic and enzymatic hydrolysis or by packaging active agent in an appropriately resistant carrier such as a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical, transdermal administration, the agents are formulated into ointments, creams, salves, powders and gels. Transdermal delivery systems can also include, e.g., patches. The present compositions can also be administered in sustained delivery or sustained release mechanisms. For example, biodegradeable microspheres or capsules or other biodegradeable polymer configurations capable of sustained delivery of the active agent can be included herein.

For inhalation, the active agent can be delivered using any system known in the art, including dry powder aerosols, liquids delivery systems, air jet nebulizers, propellant systems, and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

The active agent can be delivered alone or as pharmaceutical compositions by any means known in the art, e.g., systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

Compositions of the present disclosure may be administered via one or more routes of administration using one or more of a variety of methods known in the art. As will be appreciated by the skilled artisan, the route and/or mode of administration will vary depending upon the desired results. Selected routes of administration include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, compositions can be administered via a non-parenteral route, such as a topical, epidermal or mucosal route of administration, for example, intranasally, orally, vaginally, rectally, sublingually or topically. In one embodiment, the composition is administered by infusion. In another embodiment, the composition is administered subcutaneously.

In one particular, non-limiting example, the pharmaceutical composition may include liposomes or nanoparticles in which the active agent is disposed. In addition to other pharmaceutically acceptable carrier(s), the liposome may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference. The active agents of the present disclosure can be administered in the form of a liposome. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the active agent to be delivered. In order to cross intact mammalian skin, lipid vesicles must pass through a series of fine pores, each with a diameter less than 50 nm, under the influence of a suitable transdermal gradient. Therefore, it is desirable in certain embodiments to use a liposome which is highly deformable and able to pass through such fine pores. Liposomes can be made from phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example (but not by way of limitation), soybean PC, and egg PC. Another type is formed from mixtures of phospholipid and/or phosphatidylcholine and/or cholesterol.

In other non-limiting examples, the active agent of the present disclosure may be incorporated into particles of one or more polymeric materials, as this type of incorporation can be useful in controlling the duration of action of the active agent by allowing for controlled release from the preparations, thus increasing the half-life thereof. Non-limiting examples of polymeric materials that may be utilized in this manner include polyesters, polyamides, polyamino acids, hydrogels, poly(lactic acid), ethylene vinylacetate copolymers, copolymer micelles of, for example, PEG and poly(1-aspartamide), and combinations thereof.

In another embodiment, the active agent(s) of the present disclosure can be tableted with conventional tablet bases such as lactose, sucrose, and starch in combination with binders, such as acacia, cornstarch, or gelatin, disintegrating agents such as potato starch or alginic acid, and a lubricant such as stearic acid or magnesium stearate. Liquid preparations are prepared by dissolving the active agent(s) in an aqueous or non-aqueous pharmaceutically acceptable solvent which may also contain suspending agents, sweetening agents, flavoring agents, and preservative agents as are known in the art.

For parenteral administration, for example, the active agent(s) may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain stabilizers, preservatives, buffers, antioxidants, or other additive known to those of skill in the art.

Additional pharmaceutical methods may be employed to control the duration of action of the active agent(s). Increased half-life and controlled release preparations may be achieved through the use of polymers to conjugate, complex with, absorb, or contain the active agent(s) described herein. The controlled delivery and/or increased half-life may be achieved by selecting appropriate macromolecules (for example, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide, proteins (e. g., bovine serum albumin or human serum albumin) and the appropriate concentration of macromolecules as well as the methods of incorporation, in order to control release.

Another possible method useful in controlling the duration of action by controlled release preparations and half-life is incorporation of the active agent(s) into particles of a polymeric material such as polyesters, polyamides, polyamino acids, hydrogels, poly(lactic acid), ethylene vinylacetate copolymers, copolymer micelles of, for example, polyethylene glycol (PEG) and poly(1-aspartamide).

It is also possible to entrap the active agent(s) in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the active agent(s) is to be used as an injectable material, it can be formulated into a conventional injectable carrier. Suitable carriers include biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The active agent(s) of the present disclosure can also be administered as a pharmaceutically acceptable acid-addition or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

An effective amount of the active agent used in the treatment described herein can be determined by the attending diagnostician, as one skilled in the art, by the use of conventional techniques and by observing results obtained under analogous circumstances. In determining the therapeutically effective dose, a number of factors may be considered by the attending diagnostician, including, but not limited to: the species of the subject; its size, age, and general health; the specific condition involved; the degree of or involvement or the severity of the condition; the response of the individual subject; the particular compound administered; the mode of administration; the bioavailability characteristic of the preparation administered; the dose regimen selected; the use of concomitant medication; and other relevant circumstances.

As used herein, a “pharmaceutically-acceptable carrier, vehicle, diluent, or excipient” may also refer to a pharmaceutically-acceptable solvent, suspending agent or material for delivering the active agent(s) of the present disclosure to the subject. “Ophthalmically-acceptable vehicle, carrier, diluent, or excipient” is an ophthalmically-acceptable solvent, suspending agent, or material for delivering the active agents of the present disclosure to an eye of the subject. The carrier may be liquid or solid and is selected with the planned manner of administration in mind. Examples of pharmaceutically-acceptable vehicles, carriers, diluents, or excipients, and/or ophthalmically-acceptable vehicles, carriers, diluents, or excipients that may be utilized in accordance with the present disclosure include, but are not limited to, polyethylene glycol (PEG), polymers, carboxymethylcellulose, liposomes, ethanol, DMSO, aqueous buffers, saline solutions, solvents, oils, DPPC, lipids, and combinations thereof. Other examples include, but are not limited to, biocompatible hydrogels, bandages, and contact lenses, which can also be coated with the active agent and placed directly on the eye. The pharmaceutical compositions described or otherwise contemplated herein may further comprise at least one delivery agent that assists in delivery of the active agents to a desired site of delivery; for example but not by way of limitation, at least one delivery agent may be included in an ophthalmic composition to assist in the penetration of a surface of an eye; in certain embodiments, the delivery agent may assist in delivery to a retina of the eye. For example, in order for a topical application to be effective, the composition may need to be able to penetrate the surface of the eye so that it can travel to the desired tissue. This may include penetrating the conjunctiva and/or the cornea.

The term “topical” as used herein to define a mode of administration, means that a material is administered by being applied to an epithelial surface or tissue. In addition, as noted, the compositions of the present disclosure may be designed to provide delayed, controlled, extended, and/or sustained release using formulation techniques which are well known in the art.

As used herein, the term “concurrent therapy” is used interchangeably with the terms “combination therapy” and “adjunct therapy,” and will be understood to mean that the subject in need of treatment is treated or given another drug for the condition in conjunction with the pharmaceutical compositions of the present disclosure. This concurrent therapy can be sequential therapy, where the patient is treated first with one composition and then the other composition, or the two compositions are given simultaneously.

Another non-limiting embodiment of the present disclosure is directed to a kit that contain one or more of any of the pharmaceutical compositions described or otherwise contemplated herein. The kit may further contain a second agent as described herein above for use concurrently with the pharmaceutical composition(s). If the composition present in the kit is not provided in the form in which it is to be delivered, the kit may further contain a pharmaceutically acceptable carrier, vehicle, diluent, or other agent for mixing with the active agent for preparation of the pharmaceutical composition. The kit including the composition and/or other reagents may also be packaged with instructions packaged for administration and/or dosing of the compositions contained in the kit. The instructions may be fixed in any tangible medium, such as printed paper, or a computer-readable magnetic or optical medium, or instructions to reference a remote computer data source such as a worldwide web page accessible via the internet.

The kit may contain single or multiple doses of the pharmaceutical composition which contains the active agent. When multiple doses are present, the doses may be disposed in bulk within a single container, or the multiple doses may be disposed individually within the kit; that is, the pharmaceutical compositions may be present in the kit in unit dosage forms to facilitate accurate dosing. The term “unit dosage forms” as used herein refers to physically discrete units suitable as unitary dosages for human subjects and other mammals; each unit contains a predetermined quantity of the active agent calculated to produce the desired therapeutic effect, in association with a suitable pharmaceutical excipient. Typical unit dosage forms of liquid compositions include prefilled, premeasured ampules or syringes; for solid compositions, typical unit dosage forms include pills, tablets, capsules, or the like. In such compositions, the active agent may sometimes be a minor component (from about 0.1 to about 50% by weight, such as but not limited to, from about 1 to about 40% by weight) with the remainder being various vehicles or carriers and processing aids helpful for forming the desired dosing form.

The active agent may be provided as a “pharmaceutically acceptable salt,” which refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

The amount of the active agent that is effective in the treatment described herein can be determined by the attending diagnostician, as one of ordinary skill in the art, by the use of conventional techniques and by observing results obtained under analogous circumstances. In determining the therapeutically effective dose, a number of factors may be considered by the attending diagnostician, including, but not limited to: the species of the subject; its size, age, and general health; the specific diseases or other conditions involved; the degree, involvement, and/or severity of the diseases or conditions; the response of the individual subject; the particular active agent administered; the mode of administration; the bioavailability characteristics of the preparation administered; the dose regimen selected; the use of concomitant medication; and other relevant circumstances. A therapeutically effective amount of an active agent of the present disclosure also refers to an amount of the active agent which is effective in controlling, reducing, or ameliorating the condition to be treated.

Practice of the method of the present disclosure may include administering to a subject a therapeutically effective amount of the pharmaceutical composition (containing the active agent in any suitable systemic and/or local formulation, in an amount effective to deliver the dosages listed above. The dosage can be administered, for example, but not by way of limitation, on a one-time basis, or administered at multiple times (for example, but not by way of limitation, from one to five times per day, or once or twice per week). The pharmaceutical composition may be administered either alone or in combination with other therapies, in accordance with the inventive concepts disclosed herein.

In at least certain embodiments, the compounds of the present disclosure inhibit the activity of MAP4K4. The MAP4K4 inhibitors, such as DMX-5804 and its analogs and derivatives as described herein, may function by rescuing cell survival, mitochondrial function, and calcium cycling in cardiomyocytes. The inhibitors may be used to treating subject who have suffered strokes. In a particular embodiment, the inhibitors suppress human cardiac muscle cell death, thus can function to reduce injury due to myocardial infarct, including ischemic injury or ischemia-reperfusion injury, for example in a human heart. Additional therapeutic indications of the disclosed MAP4K4 inhibitors include neurodegeneration, skeletal muscle disorders, and cancers such as colorectal cancer (CRC), hepatocellular carcinoma (HCC), pancreatic ductal adenocarcinoma (PDAC), lung adenocarcinoma, and prostate cancer, and cancers in which inhibition of tumor cell motility is desired.

Particular cardiomyopathies that may be treated with the MAP4K4 inhibitors of the present disclosure include muscle injury, heart muscle cell injury, heart muscle cell injury due to cardiopulmonary bypass, chronic forms of heart muscle cell injury, hypertrophic cardiomyopathies, dilated cardiomyopathies, mitochondrial cardiomyopathies, cardiomyopathies due to genetic conditions, cardiomyopathies due to high blood pressure, cardiomyopathies due to heart tissue damage from a previous heart attack, cardiomyopathies due to chronic rapid heart rate, cardiomyopathies due to heart valve problems, cardiomyopathies due to metabolic disorders, cardiomyopathies due to nutritional deficiencies of essential vitamins or minerals, cardiomyopathies due to alcohol consumption, cardiomyopathies due to use of cocaine, amphetamines or anabolic steroids, cardiomyopathies due to radiotherapy to treat cancer, cardiomyopathies due to certain infections which may injure the heart and trigger cardiomyopathy, cardiomyopathies due to hemochromatosis, cardiomyopathies due to sarcoidosis, cardiomyopathies due to amyloidosis, cardiomyopathies due to connective tissue disorders, drug- or radiation-induced cardiomyopathies, idiopathic or cryptogenic cardiomyopathies, other forms of ischemic injury, including but not limited to ischemia-reperfusion injury, ischemia stroke, renal artery occlusion, and global ischemia-reperfusion injury (cardiac arrest), cardiac muscle cell necrosis, and cardiac muscle cell apoptosis.

The MAP4K4 inhibitor may be used to treat other diseases and conditions which involve the expression of a MAP4K4 protein, such as retinopathies, autoimmune diseases, inflammatory diseases (i.e., ICAM-1 related disorders, Psoriasis, Ulcerative Colitis, Crohn's disease), viral diseases (i.e., HIV, Hepatitis C), as well as cardiovascular diseases.

Particular examples of conditions that may benefit from the inhibition of MAP4K4 include, for example, the metabolic disorder known as metabolic syndrome, which is also known as syndrome X, insulin resistance syndrome, insulin-resistant hypertension, the metabolic hypertensive syndrome, and dysmetabolic syndrome. Components of the metabolic syndrome include, but are not limited to, glucose intolerance, impaired glucose tolerance, impaired fasting serum glucose, impaired fasting blood glucose, hyperinsulinemia, pre-diabetes, obesity, visceral obesity, hypertriglyceridemia, elevated serum concentrations of free fatty acids, elevated serum concentrations of C-reactive protein, elevated serum concentrations of lipoprotein(a), elevated serum concentrations of homocysteine, elevated serum concentrations of small, dense low-density lipoprotein (LDL)-cholesterol, elevated serum concentrations of lipoprotein-associated phospholipase (A2), reduced serum concentrations of high density lipoprotein (HDL)-cholesterol, reduced serum concentrations of HDL(2b)-cholesterol, reduced serum concentrations of adiponectin, adipogenesis, and albuminuria.

Compositions of the active agent can be administered in a single dose treatment or in multiple dose treatments on a schedule and over a time period appropriate to the age, weight and condition of the subject, the particular composition used, and the route of administration. In one embodiment, a single dose of the composition according to the disclosure is administered. In other embodiments, multiple doses are administered. The frequency of administration can vary depending on any of a variety of factors, e.g., severity of the symptoms, degree of immunoprotection desired, or whether the composition is used for prophylactic or curative purposes. For example, in certain embodiments, the composition is administered once per month, twice per month, three times per month, every other week, once per week, twice per week, three times per week, four times per week, five times per week, six times per week, every other day, daily, twice a day, or three times a day. The duration of treatment, e.g., the period of time over which the composition is administered, can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In one aspect, the pharmaceutical formulations comprising the active agent are incorporated in lipid monolayers or bilayers, e.g., liposomes, such as shown in U.S. Pat. Nos. 6,110,490; 6,096,716; 5,283,185; and 5,279,833. Liposomes and liposomal formulations can be prepared according to standard methods and are also well known in the art, such as U.S. Pat. Nos. 4,235,871; 4,501,728 and 4,837,028.

In one aspect, the active agent is prepared with one or more carriers that will protect the active agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

The active agent in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active agent is combined in a mixture with one or more pharmaceutically suitable excipients. Sterile phosphate-buffered saline is one example of a pharmaceutically suitable excipient.

Examples of routes of administration of the active agents described herein include parenteral injection, e.g., by subcutaneous, intramuscular or transdermal delivery. Other forms of parenteral administration include intravenous, intraarterial, intralymphatic, intrathecal, intraocular, intracerebral, or intracavitary injection. In parenteral administration, the compositions will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hanks' solution. Nonaqueous excipients such as fixed oils and ethyl oleate may also be used. An alternative excipient is 5% dextrose in saline. The excipient may contain minor amounts of additives such as substances that enhance isotonicity and chemical stability, including buffers and preservatives. These compounds can be administered by a variety of routes including, but not limited to, oral, rectal, transdermal, subcutaneous, intravenous, intramuscular, and intranasal. These compounds are effective as both injectable and oral compositions. Such compositions are prepared in a manner well known in the pharmaceutical art and comprise at least one compound having anti-cancer activity.

### Examples

The inventive concepts of the present disclosure will now be discussed in terms of several specific, non-limiting, examples. The examples described below, which include particular embodiments, will serve to illustrate the practice of the present disclosure, it being understood that the particulars shown are by way of example and for purposes of illustrative discussion of particular embodiments of the present disclosure only and are presented in the cause of providing what is believed to be a useful and readily understood description of procedures as well as of the principles and conceptual aspects of the inventive concepts.

### Experimental

All reagents and solvents were purchased from commercial suppliers and used without further purification. ¹H and ¹³C NMR spectra were recorded on a Varian spectrometer in suitable deuterated solvents. The solvents and measurement frequency used are indicated for each experiment. Signal of the residual deuterated solvent relative to tetramethylsilane was used as the internal reference. All spectra are reported in supplemental data as follows: chemical shift (in parts per million (multiplicity, coupling constant J in hertz, and number of protons). The resonance multiplicity is described as singlet (s), doublet (d), triplet (t), quartet (q), multiplet (m), doublet of doublets (dd), doublet of triplets (dt), or triplet of doublets (td). Product purity was determined by high performance liquid chromatography (HPLC). For reaction monitoring, analytical thin-layer chromatography (TLC) was performed on Merck silica gel 60 F254 strips and visualization was accomplished by irradiation with UV light (254 or 366 nm).

HPLC: HPLC was performed using an Azura P 6.1L HPLC system (Knauer, Berlin, Germany) with UV-1 detector set at k=254 nm. The samples were separated on a Sonoma C18, 10 μm, 4.6×250 mm using water-acetonitrile gradient 5-95% at 1.5 ml/min.

2-bromo-1-(4-methoxyphenyl)ethan-1-one (3): To solution a 4-hydroxyacetophenone (25 g) in dimethylformamide potassium carbonate (38 g) and 1-bromo-2-methoxyethane (30 g) were added. Reaction mixture was heated to 90° C. for 12-20 h and monitored by TLC. After completion of the reaction, the mixture was cooled to room temperature, poured into ice-water, and extracted with ethyl acetate. The organic extract was dried and evaporated. The residue was purified by flash column using hexanes/ethyl acetate (1:9). The obtained 1-(4-(2-methoxyethoxy)phenyl)ethan-1-one (71%) intermediate was brominated by bromine in diethyl ether to get 3 (90%).

1-(4-(2-methoxyethoxy)phenyl)-2-(phenylamino)ethan-1-one (4): A round-bottom flask fitted with a magnetic stir bar was charged with aniline (1.07 mol), sodium bicarbonate (1.28 mol), and N,N′-dimethylformamide (DMF, 300 mL). The slurry was stirred while adding 2-bromo-1-(4-(2-methoxyethoxy)phenyl)ethan-1-one (1.18 mol, dissolved in DMF) dropwise. The mixture was heated to 50° C. and stirred for 5 h. The reaction mixture was cooled to room temperature and poured on to ice-water to obtain a precipitate which was filtered and re-crystalized from isopropyl alcohol as light yellow solid (92% yield). ¹H NMR (400 MHz, CDCl₃) δ 7.98 (d, J=8.7 Hz, 2H), 7.23 (m, 2H), 7.00 (d, J=8.7 Hz, 2H), 6.75 (d, J=7.7 Hz, 3H), 4.57 (s, 2H), 4.20 (d, J=4.5 Hz, 2H), 3.78 (d, J=4.5 Hz, 2H), 3.46 (s, 3H).

2-amino-4-(4-(2-methoxyethoxy)phenyl)-1-phenyl-1H-pyrrole-3-carbonitrile (5): A round-bottom flask fitted with a magnetic stir bar was charged with 4 (0.31 mol), potassium hydroxide (0.94 mol) dissolved in water (50 mL), malononitrile (0.34 mol), and methanol (450 mL). The mixture was heated to 80° C. for 4 h. Afterwards the reaction mixture was cooled and evaporated to 200 mL. The precipitate was filtered and recrystallized in cold isopropyl alcohol to obtain 2-amino-4-(4-(2-methoxyethoxy)phenyl)-1-phenyl-1H-pyrrole-3-carbonitrile 5 as a brown solid (88% yield). ¹H NMR (400 MHz, CDCl₃) δ 7.54 (m, 4H), 7.41 (m, 4H), 6.95 (d, J=8.7 Hz, 2H), 4.14 (d, J=4.6 Hz, 2H), 3.76 (d, J=4.6 Hz, 2H), 3.45 (s, 3H).

5-(4-(2-methoxyethoxy)phenyl)-7-phenyl-3,7-dihydro-4H-pyrrolo[2,3-d]pyrimidin-4-one (DMX-5804): Compound 5 (0.22 mol) was dissolved in 85% formic acid (200 mL) and refluxed for 8-10 h. After cooling to room temperature, the mixture was poured onto ice-water to obtain a precipitate. The precipitate was filtered, dried, and recrystallized from dichloromethane/ethanol (1:2) to afford DMX-5804 as a white solid (80-85% yield). MP 158-160° C. ¹H NMR (400 MHz, CDCl₃) δ 12.30 (s, 1H), 7.94 (s, 1H), 7.79 (d, J=8.6 Hz, 2H), 7.63 (d, J=7.5 Hz, 2H), 7.53 (t, J=7.8 Hz, 2H), 7.41 (t, J=7.4 Hz, 1H), 7.00 (d, J=8.6 Hz, 2H), 4.17 (t, J=4.7 Hz, 2H), 3.77 (t, J=4.7 Hz, 2H), 3.46 (s, 3H). ¹³C-NMR (101 MHz, DMSO-d₆) δ 160.74, 158.09, 148.33, 143.07, 137.26, 129.79, 129.44, 127.67, 125.73, 124.80, 121.78, 121.41, 114.59, 106.35, 71.07, 67.30, 59.20.

5-(4-(2-hydroxy)phenyl)-7-phenyl-3,7-dihydro-4H-pyrrolo[2,3-d]pyrimidin-4-one (6): To a cold (−30° C.) solution of 5-(4-methoxyphenyl)-7-phenyl-3,7-dihydro-4H-pyrrolo[2,3-d]pyrimidin-4-one (300 mg) in dichloromethane was added BBr₃ (Scheme 3). The temperature of the reaction mixture was raised to room temperature by stirring for 1 h. The solvent was evaporated and to the residue was added saturated ice cold NaHCO₃ solution. The precipitate was filtered to obtain title compound 6 (286 mg, 100% yield). ¹H NMR (400 MHz, DMSO-d₆) δ 12.05 (s, 1H), 9.34 (s, 1H), 7.92 (d, J=2.99 Hz, 1H), 7.77 (d, J=8.59 Hz, 2H), 7.74 (d, J=7.55 Hz, 2H), 7.61 (s, 1H), 7.53 (t, J=7.83 Hz, 2H), 7.39 (t, J=7.39 Hz, 1H), 6.75 (d, J=8.59 Hz, 2H); ¹³C-NMR (101 MHz, DMSO-d₆) δ 159.04, 156.62, 148.09, 144.61, 137.72, 130.02, 129.58, 127.43, 124.91, 124.59, 121.53, 120.67, 115.23, 106.43.

Base-catalyzed alkylation: To a solution of compound 6 (25 g) in DMF (150 mL) was added 1-bromo-2-methoxyethane, or any alkyl halide. After stirring at 80-90° C. for 12-15 h, the reaction mixture was cooled to room temperature and poured in crushed ice-water mixture. After stirring for 1 h, precipitate was collected and re-crystalized from DCM/ethanol (1:2).

4-(4-(2-methoxyethoxy)-7-phenyl-7H-pyrrolo[2,3-d]pyrimidin-5-yl)phenol (regio-isomer 7): Crystalline white solid obtained in yield 80%; MP 221-224° C. ¹H NMR (400 MHz, DMSO-d₆) δ 9.37 (s, 1H), 8.16 (s, 1H), 7.74 (m, 4H), 7.63 (s, 1H), 7.54 (d, J=7.9 Hz, 2H), 7.41 (d, J=7.4 Hz, 1H), 6.76 (d, J=8.6 Hz, 2H), 4.16 (t, J=5.7 Hz, 2H), 3.58 (d, J=5.2 Hz, 2H), 3.24 (s, 3H). ¹³C-NMR (101 MHz, DMSO-d₆) δ 158.01, 156.68, 147.78, 147.36, 137.55, 130.16, 129.64, 127.48, 124.82, 124.45, 121.42, 121.18, 115.17, 105.60, 69.83, 58.55, 45.67.

Acidic O-Alkylation: To a mixture of compound 6 (25 g) and BF3·OEt (50 mL) was added a solution of an appropriate substrate (2-methoxyethan-1-ol, β-D-glucose pentaacetate or 1-O-acetyl-2,3,5-tri-O-benzoyl-beta-D-ribofuranose). In one condition, the solutions of substrate were prepared in dichloromethane, whereas in another condition the substrates were added solid to the reaction mixture (neat). After stirring at 25° C. for 18 h, the reaction mixture was evaporated. The residue was washed with saturated NaHCO₃ (50 mL) and extracted with chloroform (50 mL×2). The combined organic phase was dried over anhydrous MgSO₄, concentrated, and crystalized from ethanol.

7-phenyl-5-(4-(((2R,3R,4S,5S,6R)-3,4,5-trihydroxy-6-(hydroxymethyl)tetrahydro-2H-pyran-2-yl)oxy)phenyl)-3,7-dihydro-4H-pyrrolo[2,3-d]pyrimidin-4-one (8): ¹H NMR (400 MHz, DMSO-d6) δ (d, J=3.6 Hz, 1H), 7.95 (d, J=3.7 Hz, 3H), 7.75 (m, 3H), 7.55 (t, J=5.28 Hz, 2H), 7.41 (t, J=7.42 Hz, 1H), 7.04 (dd, J=2.94 Hz, 2H), 4.88 (d, J=7.39 Hz, 1H), 3.96 (bs, 5H), 3.70 (dd, J=5.28 Hz, 1H), 3.47 (dd, J=5.86 Hz, 1H), 3.34 (m, 1H), 3.26 (m, 2H), 3.17 (t, J=3.17 Hz, 1H).

5-(4-((3,4-dihydroxy-5-(hydroxymethyl)tetrahydrofuran-2-yl)oxy)phenyl)-7-phenyl-3,7-dihydro-4H-pyrrolo[2,3-d]pyrimidin-4-one (9): ¹H NMR (400 MHz, DMSO-d6) δ 12.11 (d, J=3.6 Hz, 1H), 7.95 (d, J=3.7 Hz, 1H), 7.89 (d, J=8.7 Hz, 2H), 7.76 (d, J=7.4 Hz, 2H), 7.71 (s, 1H), 7.54 (t, J=7.9 Hz, 2H), 7.41 (t, J=7.4 Hz, 1H), 6.98 (d, J=8.8 Hz, 2H), 5.48 (s, 1H), 5.31 (s, 1H), 4.98 (s, 1H), 4.69 (s, 1H), 4.02 (s, 2H), 3.89 (m, 1H), 3.55 (dd, J=5.0 Hz, 1H), 3.37 (m, 1H).

MAP4K4 inhibition assay: MAP4K4 assay for IC₅₀ determination was performed using a kit by BioAssay Systems Services (Hayward, CA). Briefly, MAP4K4 in assay buffer (40 ng in 10 μL) was incubated with varying concentrations of test compounds (5 μL in dimethylsulfoxide) for 15 min at 37° C. The compounds were titrated from 900 nM (final reaction concentration) in 3× steps. Compound dilutions were made in assay buffer with 0.036% dimethylsulfoxide. Reactions were initiated by adding 5 μL reaction mix (2.6 μg/μL MBP, 193 μM ATP; final reaction concentration of 13 μg MBP, 48 μM ATP) and run at 37° C. for 20 min. These kinase reactions were stopped with an ADP detection reagent and fluorescence was measured at 530_{Ex}/590_{Em}. IC50 was computed using GraphPad Prism Nonlinear Sigmoidal Dose-Response fitting.

### Results

Original process for synthesis of DMX-5804: Synthesis of DMX-5804 via the original discovery route of Fiedler et al., is shown in Scheme 1 below. The researchers used Chan-Lam like amination with excessive copper (II) acetate monohydrate and 2-phenyl-1, 3,2-dioxoborinone (commercially not available and no synthetic scheme reported) at 60° C. for 24 h. Following the required work-up, crude compound was purified by reverse-phase preparative HPLC-MS to afford 4-chloro-5-iodo-7-phenyl-7H-pyrrolo[2,3-d]pyrimidine (1) in 33% yield. The chloropyrimidine derivative was converted to 5-iodo-7-phenyl-3,7-dihydro-4H-pyrrolo[2,3-d]pyrimidin-4-one (2) by refluxing in acetic acid in presence of sodium acetate for h, reportedly with 97% yield. Compound 2 was coupled with 2-(4-(2-methoxyethoxy)phenyl-4,4,5,5-tetramethyl-1,3,2-dioxaborolan by using microwave assisted Suzuki-Miyaura cross-coupling reaction at 120° C. for 3 h in presence of Pd(dppf)Cl₂. The resultant DMX-5804 was reported at 18% yield. The overall yield of the Fiedler et al., method was reported as 22%. Importantly, all stages required purification by preparative HPLC.

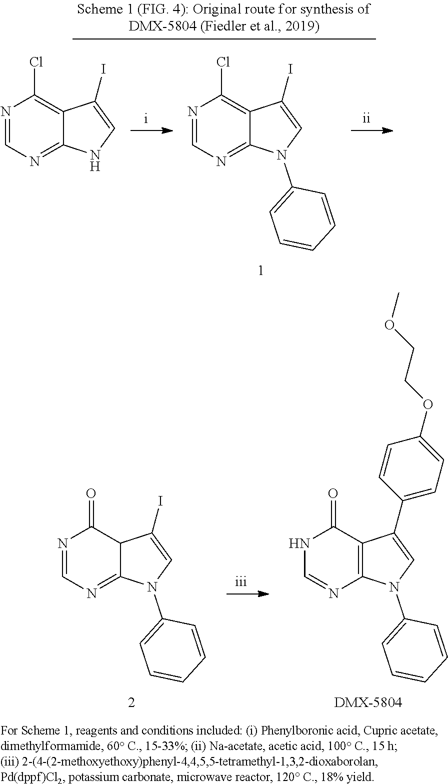

In the present work, reproduction of the Fiedler et al., route for synthesis of DMX-5804 was attempted, however we faced several obstacles in the process. First, we couldn't find any commercial source for 2-phenyl-1, 3,2-dioxoborinone and no reference was available to make this intermediate. To overcome this issue, we performed the stage 1 reaction with phenylboronic acid as a substitute for phenyl dioxoborinone and used copper (II) acetate monohydrate in DMF at 60° C. However, this strategy yielded only trace amounts of product even after 24 h. Arnold, Lee D. et al reported the same reaction for the synthesis of 1 using phenylboronic acid in dichloromethane, but the reaction duration was 12 d at room temperature, which is time-consuming and not meant for commercial production purposes (Arnold, L. D., et al., 6,6-bicyclic ring substituted heterobicyclic protein kinase inhibitors. 2012, OSI Pharmaceuticals, LLC (Farmingdale, NY): USA). With trace amounts of 1 in hand, we proceeded further to synthesize 2 using the conditions reported in the Fiedler et al., method. This reaction proceeded smoothly and afforded excellent yields of 2 (>90% yield). However, the microwave reaction in stage iii (Scheme 1) to synthesize DMX-5804, was again challenging. We observed significant charring and the desired compound was in trace amounts (<5%). We tried this reaction without microwave assistance, but no product formation occurred. We used a conventionally available kitchen microwave for this reaction, which might have had a significant bearing on the way reaction proceeded. Regardless, the reaction appears to require substantial energy input to drive this reaction forward. Based on these experiences, we decided to establish a de novo synthetic route for DMX-5804.

De novo synthesis of DMX-5804: The Fiedler et al., route for synthesis of DMX-5804 appears to be capable of supplying medicinal chemists with quantities sufficient for initial in vitro and in vivo studies, however this route is inadequate to supply meaningful and commercial amounts of DMX-5804 in a reproducible manner. We identified following drawbacks of the Fiedler et al., method, which make it very challenging, if not impossible, to translate into a commercial scale: (a) the requirement of transition metal catalysts, (b) high cost of boronate intermediate, (c) microwave-assisted reaction, (d) need of preparative HPLC for purification of intermediates and the final product, (e) poor yields with reproducibility challenges in cross-coupling reactions, and (f) safety concerns in discarding metallic complexes.

Because of the high cost, and inaccessibility of boronates in step 1 of Scheme 1, we sought to start synthesis from readily-available and inexpensive starting materials. As shown in Scheme 2, we identified substituted 2-bromo-1-(4-(2-methoxyethoxy)phenyl)ethan-1-one (3) as a suitable starting material for DMX-5804. It was reacted with aniline in DMF at room temperature in presence of mild base for 3 h to produce 1-(4-(2-methoxyethoxy)phenyl)-2-(phenylamino)ethan-1-one (4) in quantitative yields (90%). The product was recrystallized in isopropyl alcohol. In the next step, a pyrrole derivative (5) was synthesized via Knoevenagel condensation of malononitrile. We screened diisopropylethylamine, trimethylamine, KOH, and potassium-tert-butoxide as bases for this reaction. The reaction with KOH provided >95% of 5, with 80-86% isolated yield; the other bases yielded approximately 70% conversion.

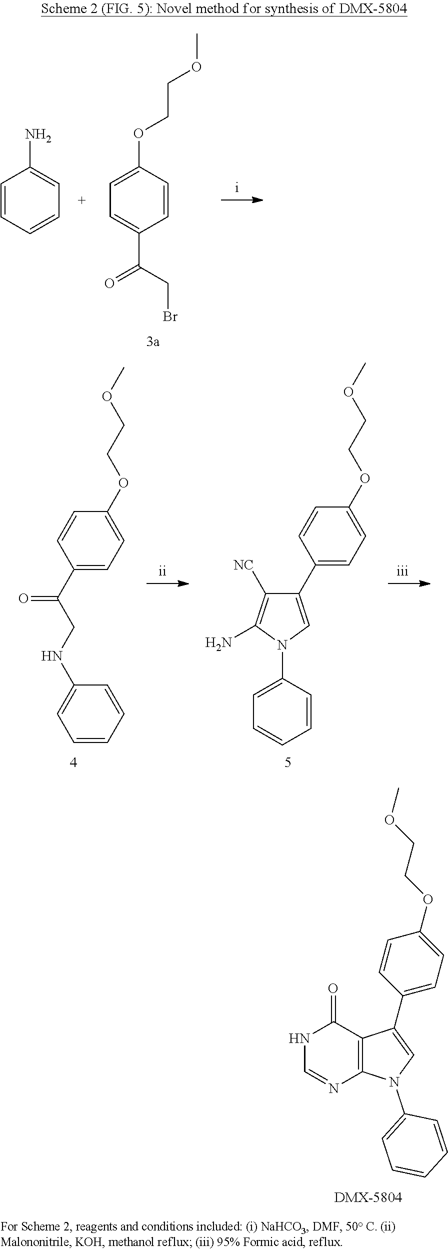

With 2-amino-pyrrole-3-carbonitrile derivative (5) in hand, we turned our focus to the assembly of pyrimidine ring in the structure of DMX-5804. Compound 5 was refluxed in 95% formic acid for 8-10 h. After cooling, the reaction mixture was poured into ice-water to precipitate the crude compound. The crude compound was dissolved in a mixture of chloroform/methanol (1:5), decolorized with activated charcoal, and re-crystallized from chloroform/methanol mixture to obtain DMX-5804 with 99% purity as analyzed by HPLC (FIG. 2). It is noteworthy that the reaction mixture and conditions did not impact the stability of DMX-5804. This observation is in contrast to the Fiedler et al., method where significant charring and loss of final product was found.

General new route for synthesis of DMX-5804 and its analogs: After obtaining reproducible yields of DMX-5804 from the Scheme 2 reaction, we turned to generalizing this scheme to synthesize novel analogs of DMX-5804. We modified O-alkoxy functionality because modification in any other place in the structure of DMX-5804 has been reported detrimental to the purported MAP4K4 inhibitory activity. Here, we replaced compound 3a (of Scheme 2) with 2-bromo-1-(4-methoxyphenyl)ethan-1-one (3b in Scheme 3) to obtain a compound with phenolic functional group (6) which is hypothesized to serve as an intermediate for synthesis of DMX-5804 or its analogs (Scheme 3).

First, we attempted base-catalyzed O-alkylation in a variety of conditions (Table 1). Initially we performed the reaction in acetonitrile using potassium carbonate at 60° C. for 12 h, which resulted in only trace amounts of DMX-5804, but approximately 20% compound 7 (pyrimidine O-alkylation), and unreacted starting material. We also screened various solvents combined with different bases to get DMX-5804. The reaction involving potassium carbonate in DMF resulted in the quantitative consumption of the starting material. However, DMX-5804 was obtained only in small amounts (5-7%), whereas compound 7 was isolated as the major product (88%). To check the stability of compound 7, we refluxed it in dioxane-HCl for 6 h. Compound 7 was stable in acidic conditions and its solubility in polar solvents was significantly better than DMX-5804. Also, its HPLC retention time was identical to that of DMX-5804 (data not shown).

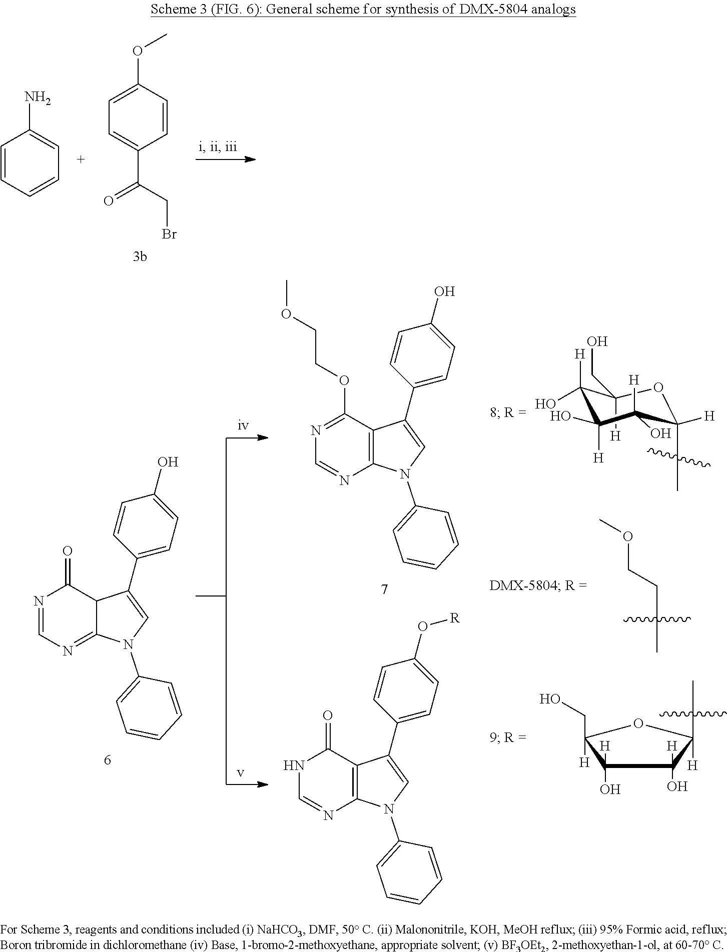

TABLE 1
Reaction conditions employed for base-catalyzed
and acid catalyzed synthesis of DMX-5804.

| | | | Time | Temp | DMX-5804 | |
|---|---|---|---|---|---|---|
| Entry | Solvent | Base/Acid | (h) | (° C.) | (%) | 7 (%) |
| 1 | Acetonitrile | K₂CO₃ | 12 | reflux | Trace | 20 |
| 2 | Acetone | K₂CO₃ | 15 | reflux | Trace | <10 |
| 3 | DMF | K₂CO₃ | 12 | 80 | 5-7 | 88 |
| 4 | DMF | CsCO₃ | 12 | 80 | 10 | 70 |
| 5 | DMSO | K₂CO₃ | 15 | 60 | Trace | 55 |
| 6 | DMF | TEA | 15 | 80 | Trace | 60 |
| 7 | THF | NaH | 5 | 10 | Trace | 20 |
| 8 | DMF | NaH | 8 | 10 | Trace | 15 |
| 9 | DCM | BF₃OEt | 10-12 | 40 | 25-30 | — |
| 10 | — | BF₃OEt | 10-12 | 60 | 60-70 | — |
| 11 | Methyl glycol | BF₃OEt | 6-8 | 70-80 | 80-85 | — |

For entries 1-8, 1-bromo-2-methoxyethane was used as a reactant; for entries 9-10, 2-methoxyethan-1-ol was used as a reactant (Scheme 3).

In order to shift alkylation to desired phenolic site, we changed reaction conditions to acidic by using boron trifluoride diethyl etherate as a Lewis acid (entries 9-11 of Table 1). We found that in presence of boron trifluoride diethyl etherate (BF₃OEt₂) in dichloromethane the reaction resulted in phenolic alkylation (entry 9). Hypothesizing that the relatively low separated yields of this reaction are due to the insolubility of phenolic derivative in dichloromethane, we decided to perform this reaction in neat conditions with BF₃OEt₂. This modification produced satisfactory yields of DMX-5804 (entry 10). Further, we added excess of methylglycol as a solvent as well as a reactant in presence of BF₃OEt₂ to obtain quantitative yields of DMX-5804 in good time (entry 11). In these acidic conditions, the other regioisomer (pyrimidine alkylated compound 7) was completely absent. Previously, the use of BF₃OEt₂ for phenolic alkylation has been reported. In addition to the compound DMX-5804, we employed this route for synthesis of ribose- and glucose-conjugated analogs. These sugar analogs were particularly interesting because of their increased solubility in aqueous medium compared to virtually insoluble DMX-5804. Various 0-conjugated analogs could be made using Scheme 3 via ether or ester linkages to various compounds, including but not limited to: (a) carbohydrates, such as alcohols, alditols, glycols, polyols, monosaccharides, disaccharides, oligosaccharides, polysaccharides, and glycosylamines (amino sugars), (b) amino acids, such wild-type and non-wild type amino acids, include L and D forms thereof, (c) peptides, peptide aptamers, and proteins comprising the amino acids of (b) in any length, (d) nucleobases such as adenine, thymine, cytosine, uracil, and guanine, and nucleosides and nucleotides thereof, (e) oligonucleotide aptamers, peptide nucleic acids, ribonucleic acids, deoxyribonucleic acids, and (f) phosphate and sulfonyl moieties.

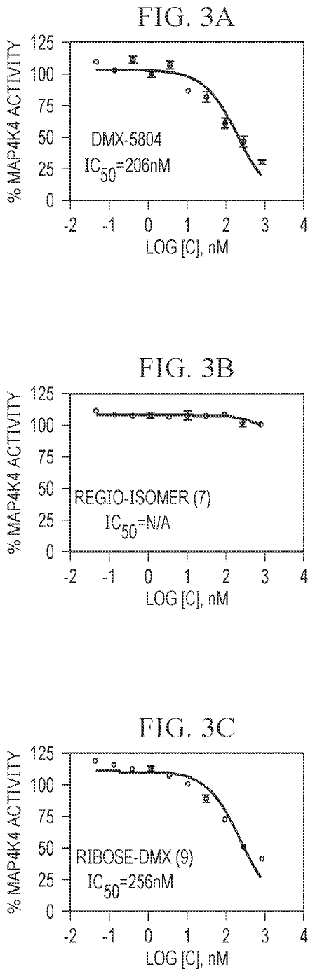

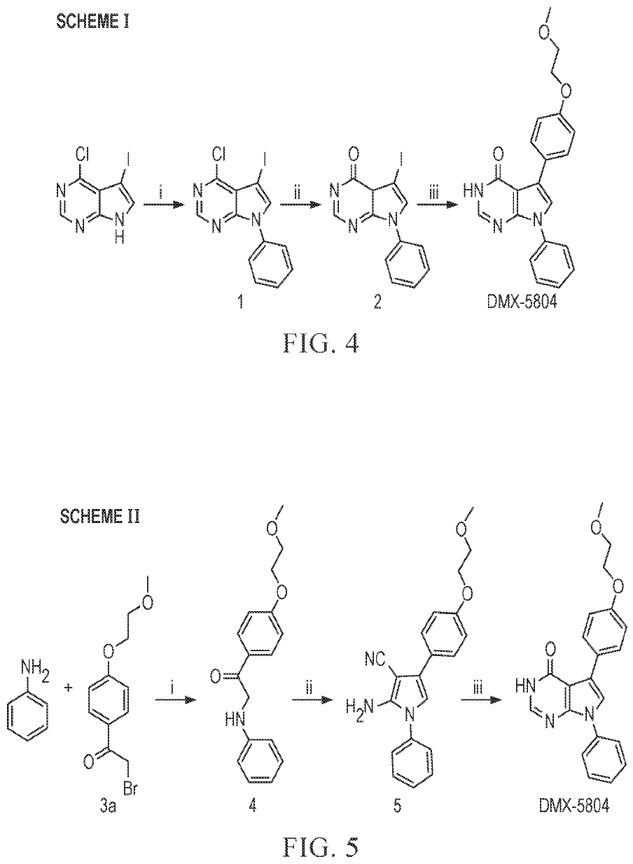

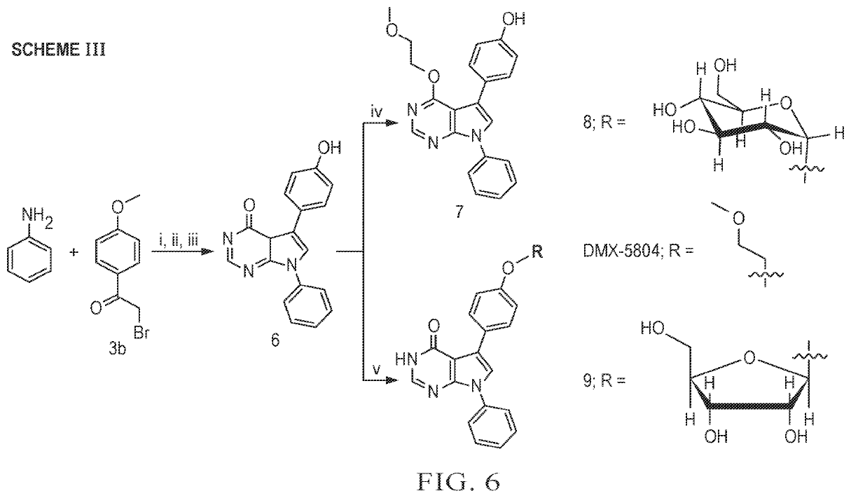

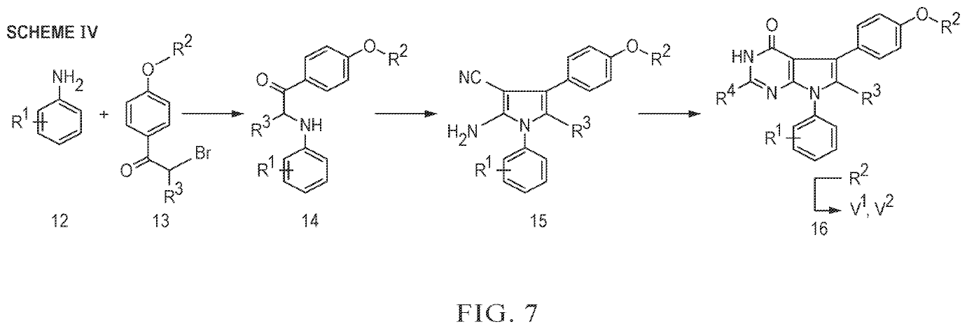

MAP4K4 inhibition assay: The MAP4K4 inhibitory activity of DMX-5804, Ribose-analog 9, and regio-isomer 7 were examined in a cell free assay using recombinant MAP4K4 enzyme and myelin basic protein (MBP) as its substrate. The results of the assay are given in FIG. 3. We found that concentration for 50% inhibition of MAP4K4 (IC₅₀) of DMX-5804 was 205.9 nM. Compound 7, where the alkoxy group was O-conjugated in pyrimidine ring, showed no inhibitory activity in the concentration range tested. However, ribose-analog 9 showed inhibitory activity (IC₅₀=255.8 nM) comparable to that of DMX-5804. These data demonstrate that it is possible to modify phenolic group for targeting purpose and/or to alter pharmacokinetic and pharmacodynamic profile of this class.

As described above in Scheme 3, step (v) results in the production of an analog having the chemical structure I:

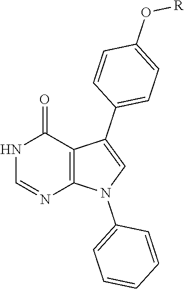

wherein R is selected from (a) carbohydrates, such as alcohols, alditols, glycols, polyols, monosaccharides, disaccharides, oligosaccharides, polysaccharides, and glycosylamines (amino sugars), (b) amines and amino acids, such wild-type and non-wild type amino acids, include L and D forms thereof, (c) peptides, peptide aptamers, and proteins comprising the amino acids of (b) in any length, (d) nucleobases such as adenine, thymine, cytosine, uracil, and guanine, and nucleosides and nucleotides thereof, (e) oligonucleotide aptamers, peptide nucleic acids, ribonucleic acids, deoxyribonucleic acids, and (f) phosphate and sulfonyl moieties.

The new process described above (Scheme 2) to synthesize DMX-5804 was effectively used for a large-scale manufacturing process which generated API grade (>99.99 by HPLC area %) in excellent (80-85%) yield. In this novel synthetic route, 1-(4-(2-methoxyethoxy)phenyl)-2-(phenylamino)ethan-1-one (1) eliminated the need of using a transition metal catalyst and expensive borates. In addition, this intermediate made it easy to access various DMX-5804 analogs. In comparison, the initial discovery route of Fiedler et al., 2019, generated DMX-5804 in only 22% yield. Notably, the presently disclosed novel three-step sequence did not require purification steps using column chromatography. The general scheme disclosed herein also eliminates the use of anhydrous solvents and microwave-assisted reaction step, which is very conducive to commercial scale synthesis of DMX-5804. Scheme 3 was created to synthesize novel analogs and derivatives and analogs of DMX-5804.

Scheme 4 shows another synthetic pathway for making DMX-5804 derivatives and analogs. Compound 16 is a generic structure, and compounds 17-54 are specific but non-limiting examples of analogs that can be formed via a synthetic pathway similar to Scheme 4 when the appropriate precursor molecules are substituted for those shown in Scheme 4.

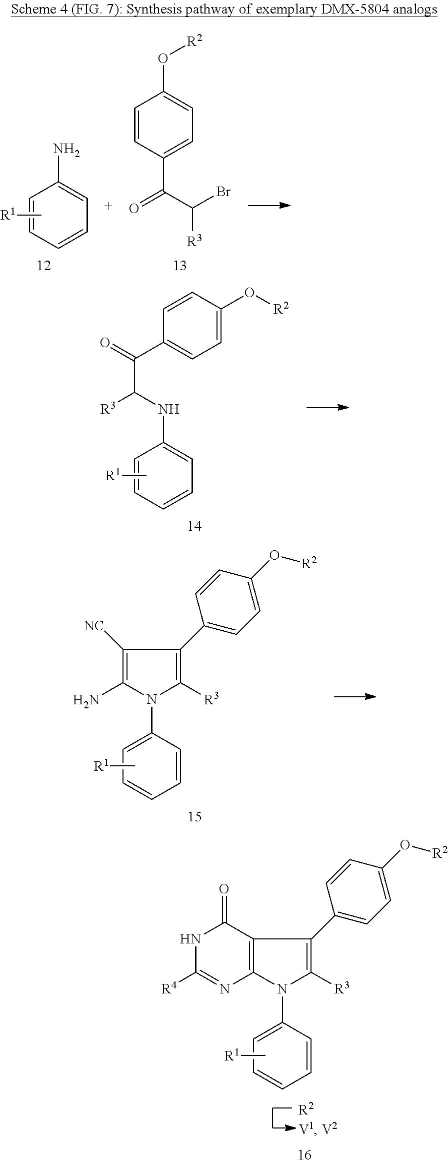

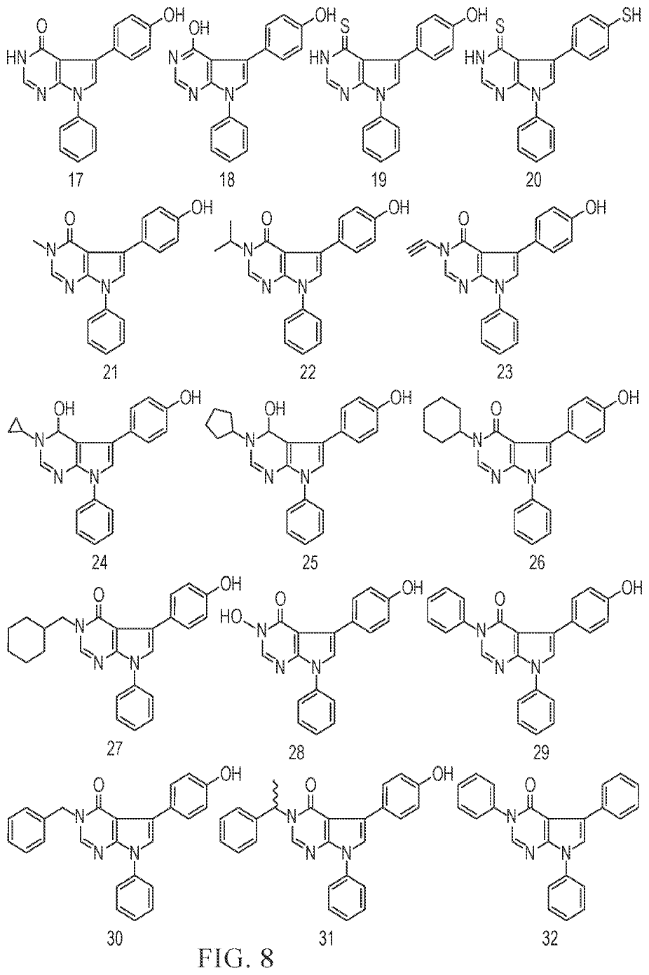

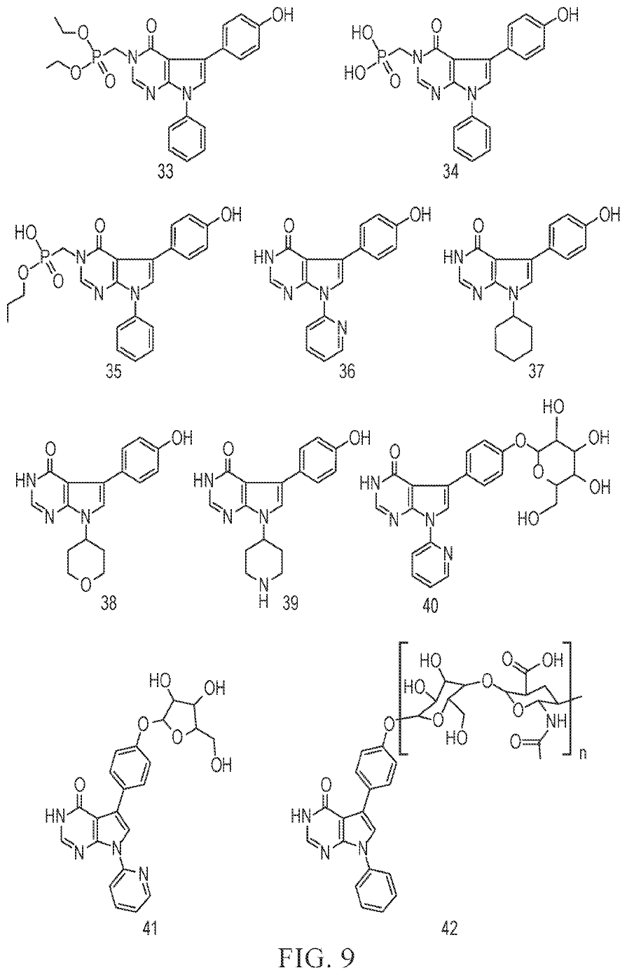

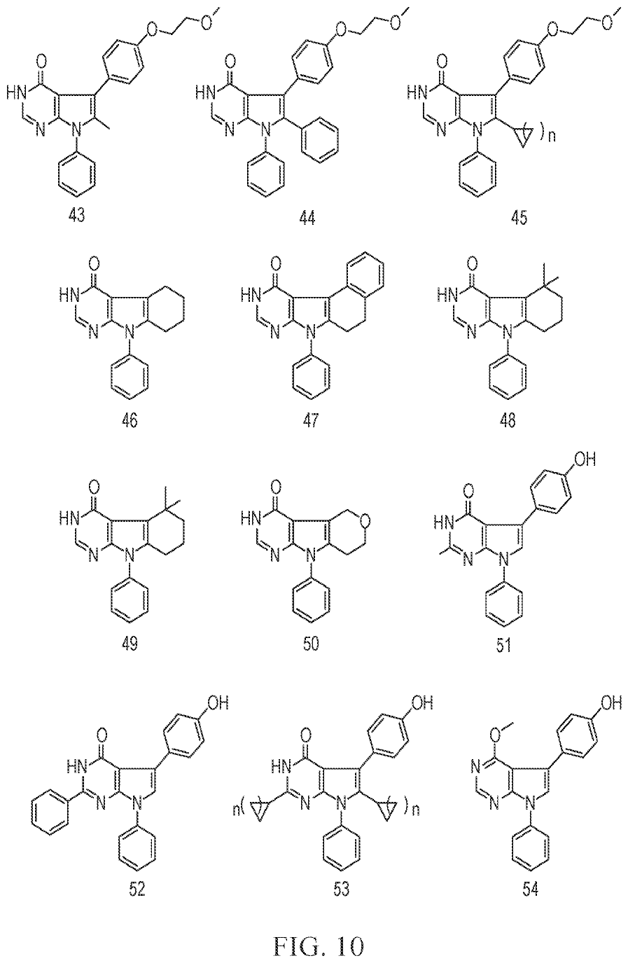

Non-limiting examples of particular DMX-5804 analogs of the present disclosure are shown below, and depicted in FIG. 8, FIG. 9, and FIG. 10 (in 42 n=1-10; in 45 and 53 n=1-8).

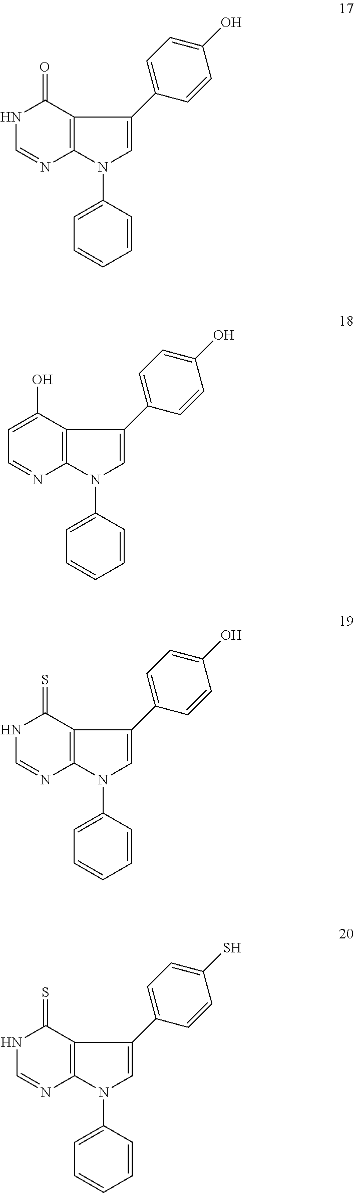 [SMILES: O=c1[nH]cnc2c1c(-c1ccc(O)cc1)cn2-c1ccccc1 ; Oc1ccc(-c2cn(-c3ccccc3)c3nccc(O)c23)cc1 ; Oc1ccc(-c2cn(-c3ccccc3)c3nc[nH]c(=S)c23)cc1 ; S=c1[nH]cnc2c1c(-c1ccc(S)cc1)cn2-c1ccccc1]

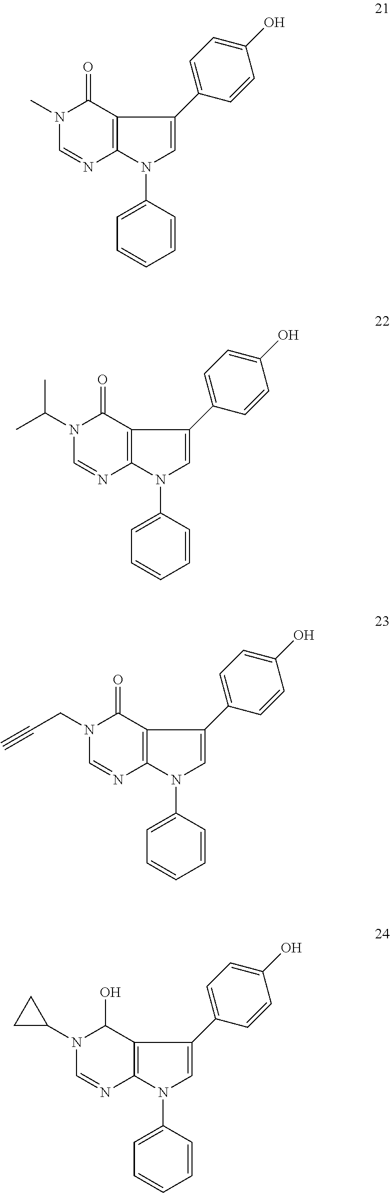 [SMILES: Cn1cnc2c(c(-c3ccc(O)cc3)cn2-c2ccccc2)c1=O ; CC(C)n1cnc2c(c(-c3ccc(O)cc3)cn2-c2ccccc2)c1=O ; C#CCn1cnc2c(c(-c3ccc(O)cc3)cn2-c2ccccc2)c1=O ; Oc1ccc(-c2cn(-c3ccccc3)c3c2C(O)N(C2CC2)C=N3)cc1]

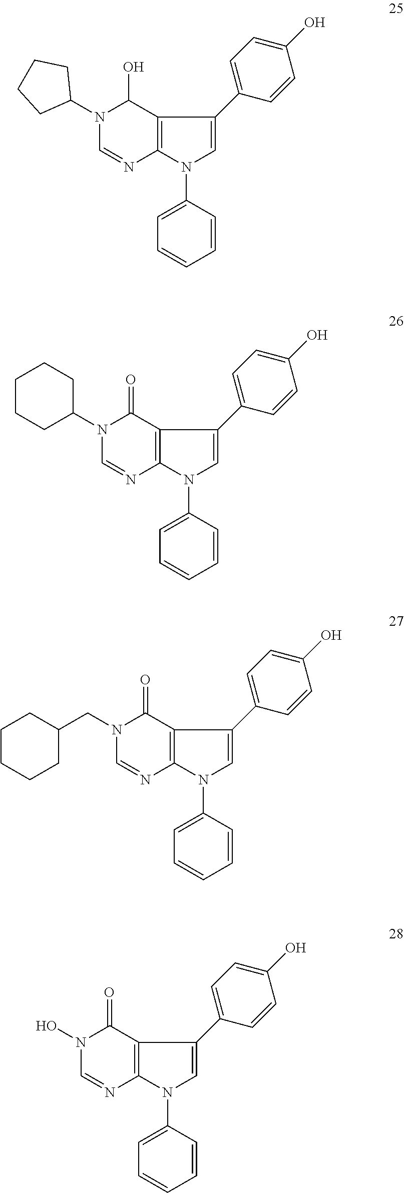 [SMILES: Oc1ccc(-c2cn(-c3ccccc3)c3c2C(O)N(C2CCCC2)C=N3)cc1 ; O=c1c2c(-c3ccc(O)cc3)cn(-c3ccccc3)c2ncn1C1CCCCC1 ; O=c1c2c(-c3ccc(O)cc3)cn(-c3ccccc3)c2ncn1CC1CCCCC1 ; O=c1c2c(-c3ccc(O)cc3)cn(-c3ccccc3)c2ncn1O]

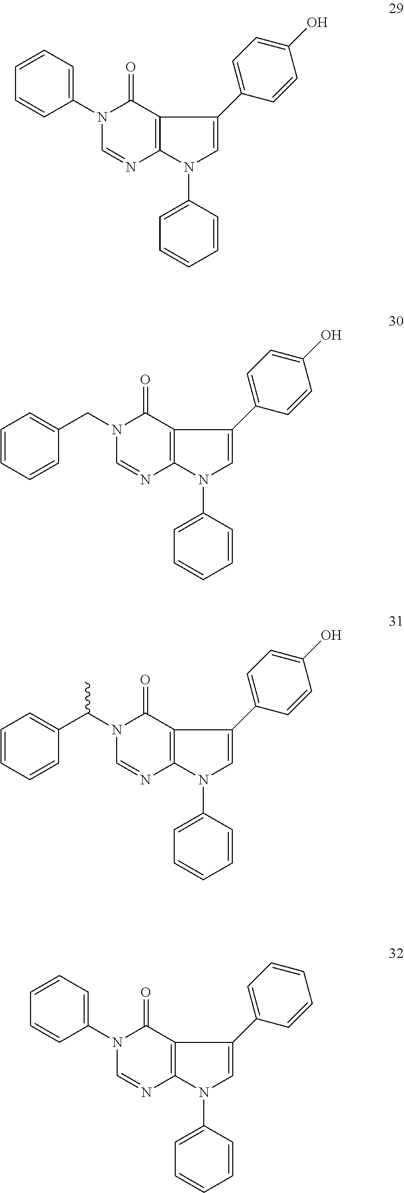 [SMILES: O=c1c2c(-c3ccc(O)cc3)cn(-c3ccccc3)c2ncn1-c1ccccc1 ; O=c1c2c(-c3ccc(O)cc3)cn(-c3ccccc3)c2ncn1Cc1ccccc1 ; CC(c1ccccc1)n1cnc2c(c(-c3ccc(O)cc3)cn2-c2ccccc2)c1=O ; O=c1c2c(-c3ccccc3)cn(-c3ccccc3)c2ncn1-c1ccccc1]

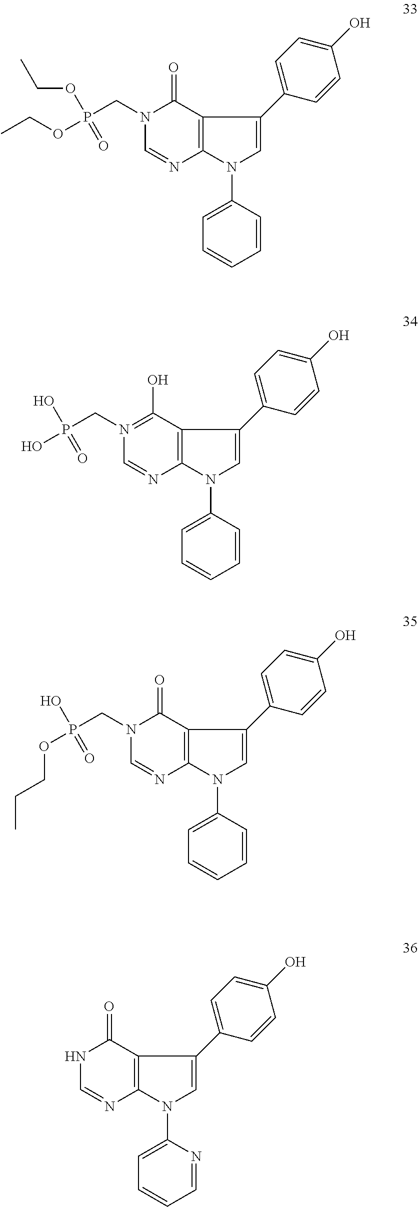

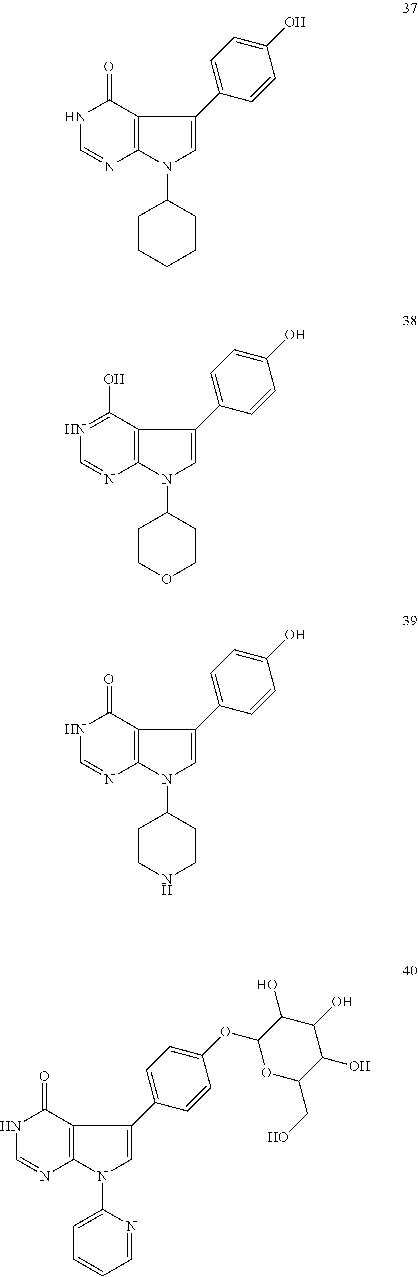 [SMILES: O=c1[nH]cnc2c1c(-c1ccc(O)cc1)cn2C1CCCCC1 ; Oc1ccc(-c2cn(C3CCOCC3)c3ncnc(O)c23)cc1 ; O=c1[nH]cnc2c1c(-c1ccc(O)cc1)cn2C1CCNCC1 ; O=c1[nH]cnc2c1c(-c1ccc(OC3OC(CO)C(O)C(O)C3O)cc1)cn2-c1ccccn1]

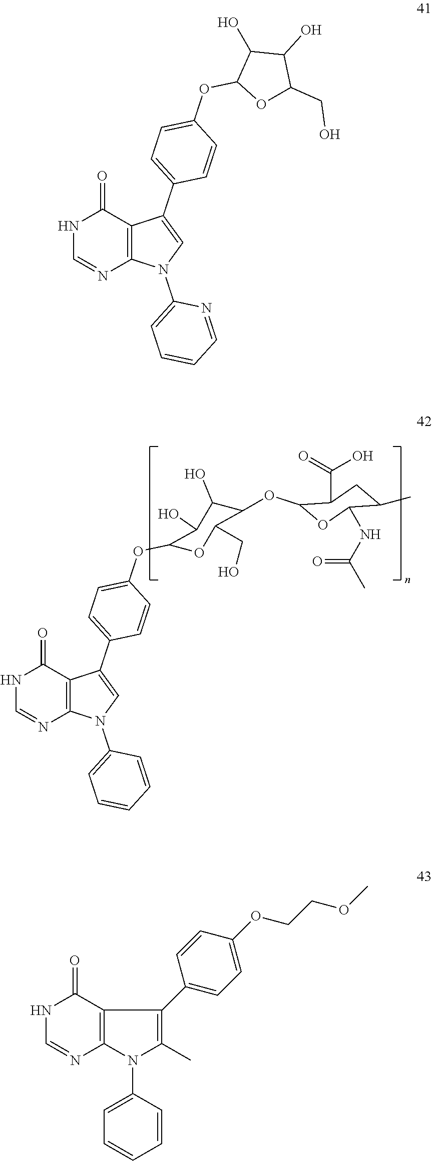

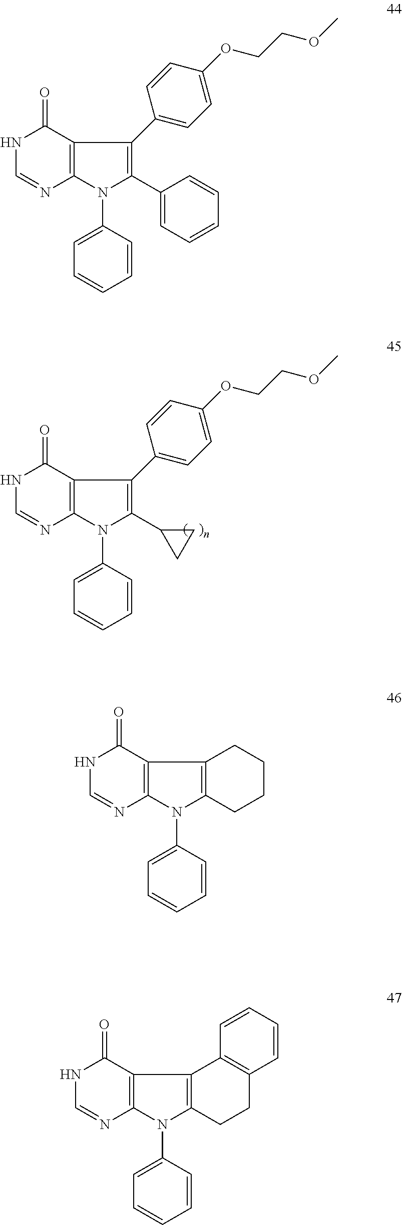

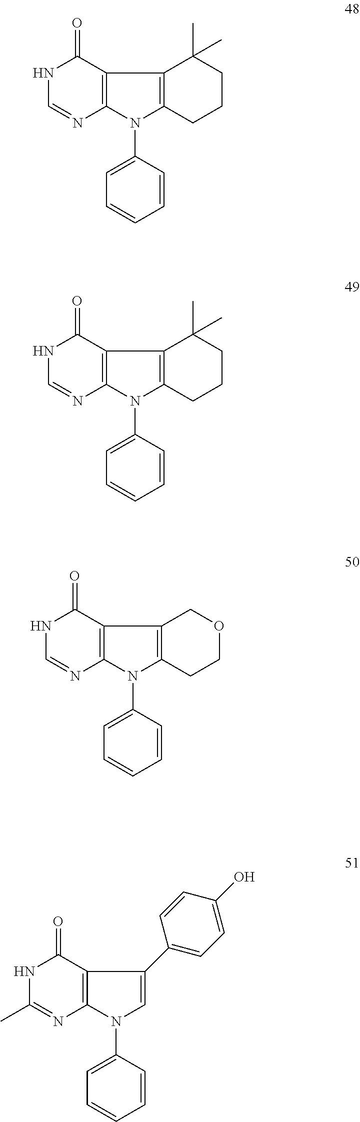

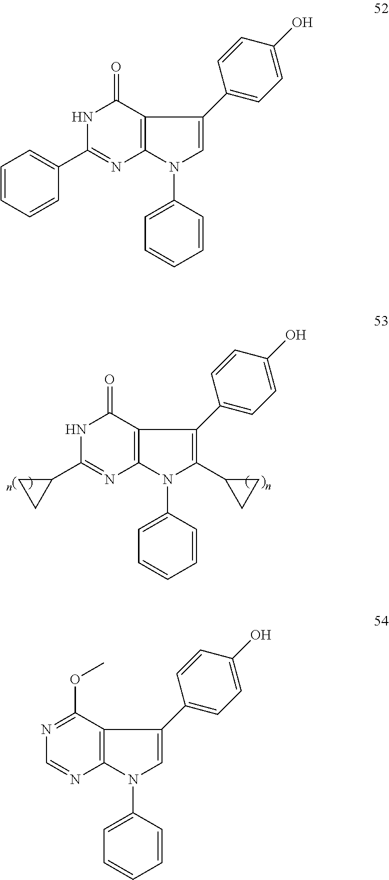

### Dmx-5804 Derivatives and Analogs

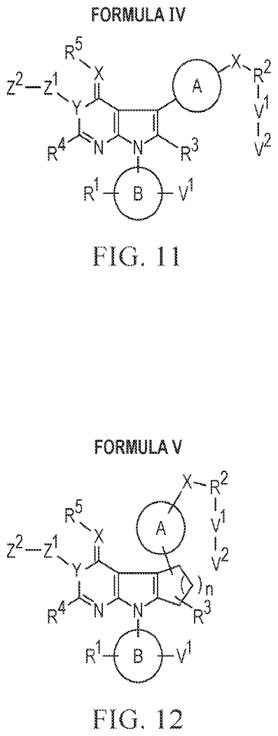

Shown below and depicted in FIG. 11 is a generic chemical structure (Formula IV) of DMX-5804 derivatives and analogs which are intended for use as MAP4K4 inhibitors according to the methods of the present disclosure.

In certain embodiments, the present disclosure is directed to compounds having Formula IV, or pharmaceutically acceptable salts thereof:

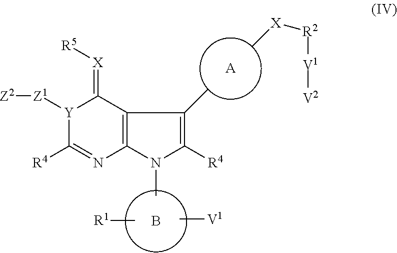

wherein:
X is selected from O, OH, and S;
Y is selected from N and NH;
R¹ is selected from H, deuterium (D), halo, —CN and C₁₋₆ alkyl, monosaccharides, disaccharides, oligosaccharides, polysaccharides, and polyethylene glycols comprising repeating units of (OCH₂CH₂)ₙ, where n=1-3000;
R² is absent, or is selected from H, D, halo, —CN and C₁₋₆ alkyl, monosaccharides, disaccharides, oligosaccharides, polysaccharides, and polyethylene glycols comprising repeating units of (OCH₂CH₂)ₙ, where n=1-3000;
R³ is selected from H, borane (B), halo, C₁₋₆ alkyl, C₂₋₆ alkenyl, C₁₋₆ haloalkyl, —NRR, —OR, 5 or 6 membered heteroaryl rings, and 3 to 8 membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀);
R⁴ is selected from H, borane (B), halo, C₁₋₆ alkyl, C₂₋₆ alkenyl, C₁₋₆ haloalkyl, —NRR, —OR, 5- or 6-membered heteroaryl rings, and 3- to 8-membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀);
R⁵ is selected from H, borane (B), halo, C₁₋₆ alkyl, C₂₋₆ alkenyl, C₁₋₆ haloalkyl, —NRR, —OR, P(O)(OH)₂, P(O)(O—C₁₋₆alkyl))₂, 5- or 6-membered heteroaryl rings, and 3- to 8-membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀), or is absent when X is O;
Z¹ is selected from H, C, O, P, C₁₋₁₀ alkyl, phenyl, substituted phenyl, and benzyl;
Z² is absent or is selected from —NR—, —O—, —C(O)—, —SO₂—, —SO₂NR—, —NRSO₂—, —C(O)NR—, —NRC(O)—, —C(O)O—, and —NRC(O)NR;
V¹ is absent or is selected from ¹²C, ¹³C, H, D, halo, —CN, C₁₋₆ alkyl, monosaccharides, disaccharides, oligosaccharides, polysaccharides, polyethylene glycols comprising repeating units of (OCH₂CH₂)ₙ, where n=1-3000;
V² is selected from heterocycloalkyl rings, heteroaryl rings, ¹²C, ¹³C, H, D, B, halo, —CN, C₁₋₆ alkyl, monosaccharides, disaccharides, oligosaccharides, polysaccharides, polyethylene glycols comprising repeating units of (OCH₂CH₂)ₙ, where n=1-3000, C₂₋₆ alkenyl, C₁₋₆ haloalkyl, —NRR, —OR, wherein the heteroaryl rings are 5- or 6-membered, the heterocycloalkyl rings are 3- to 8-membered, and wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴; and cyclo-alkyls (C₁₋₁₀);
ring A is selected from aromatic, heteroaryl, and cycloaryl rings, wherein the aromatic and heteroaryl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cycloalkyls (C₁₋₁₀), and wherein the cycloalkyl ring is an unsubstituted heterocycloalkyl ring or a heterocycloalkyl ring substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cyclo-alkyls (C₁₋₁₀);
ring B is selected from heteroaryl, aromatic, and cycloaryl rings, wherein the aromatic and heteroaryl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cycloalkyls (C₁₋₁₀), and wherein the cycloalkyl ring is an unsubstituted heterocycloalkyl ring or a heterocycloalkyl ring substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cyclo-alkyls (C₁₋₁₀); and
R is selected from H, D, B, halo, —CN and C₁₋₆ alkyl, C₂₋₆ alkenyl, C₁₋₆ haloalkyl, monosaccharides, disaccharides, oligosaccharides, polysaccharides, polyethylene glycols comprising repeating units of (OCH₂CH₂)ₙ, where n=1-3000, OR¹, OR⁴, NR¹R¹, NR⁴R⁴, NR¹R⁴, 5- or 6-membered heteroaryl rings, and 3- to 8-membered heterocycloalkyl rings, wherein the heteroaryl and heterocycloalkyl rings are unsubstituted, or are substituted with 1-4 groups selected from: oxo, halo, OR¹, OR⁴, NR¹R¹, NR⁴R⁴, NR¹R⁴, C₁₋₆ alkyl, C₁₋₆ alkyl substituted with OR¹, OR⁴, C₁₋₆ alkyl substituted with NR¹R¹, NR⁴R⁴, or NR¹R⁴, C₁₋₆ alkyl substituted with —C(O)R¹, —C(O)R⁴, —NR¹C(O)R¹, —NR⁴C(O)R⁴, NRIC(O)R⁴, or —NR⁴C(O)R¹; and cycloalkyls (C₁₋₁₀).

In certain embodiments of the above Formula IV, when Z¹ is H, at least one of R³ and R⁴ is not H or D, or when Z¹ or Z² comprise a P, at least one of R³ and R⁴ is not H or D.

Shown below and depicted in FIG. 12 is a generic chemical structure (Formula V) of DMX-5804 derivatives and analogs which are intended for use as MAP4K4 inhibitors according to the methods of the present disclosure.

In certain embodiments, the present disclosure is directed to compounds having Formula V, or pharmaceutically acceptable salts thereof:

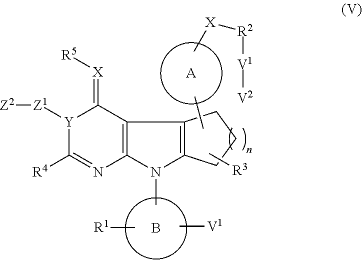

wherein:
X is selected from O, OH, and S;
Y is selected from N and NH;
n is 1, 2, 3, 4, 5, 6, 7 or 8;
R¹ is selected from H, deuterium (D), halo, —CN and C₁₋₆ alkyl, monosaccharides, disaccharides, oligosaccharides, polysaccharides, and polymers thereof, and polyethylene glycols comprising repeating units of (OCH₂CH₂)ₙ, where n=1-3000;
R² is absent, or is selected from H, D, halo, —CN and C₁₋₆ alkyl, monosaccharides, disaccharides, oligosaccharides, polysaccharides, and polyethylene glycols comprising repeating units of (OCH₂CH₂)ₙ, where n=1-3000;
R³ is selected from H, borane (B), halo, C₁₋₆ alkyl, C₂₋₆ alkenyl, C₁₋₆ haloalkyl, —NRR, —OR, 5 or 6 membered heteroaryl rings, and 3 to 8 membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀);
R⁴ is selected from H, borane (B), halo, C₁₋₆ alkyl, C₂₋₆ alkenyl, C₁₋₆ haloalkyl, —NRR, —OR, 5- or 6-membered heteroaryl rings, and 3- to 8-membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀);
R⁵ is selected from H, borane (B), halo, C₁₋₆ alkyl, C₂₋₆ alkenyl, C₁₋₆ haloalkyl, —NRR, —OR, P(O)(OH)₂, P(O)(O—C₁₋₆alkyl))₂, 5- or 6-membered heteroaryl rings, and 3- to 8-membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀), or is absent when X is O;
Z¹ is selected from H, C, O, P, C₁₋₁₀ alkyl, phenyl, substituted phenyl, and benzyl;
Z² is absent or is selected from —NR—, —O—, —C(O)—, —SO₂—, —SO₂NR—, —NRSO₂—, —C(O)NR—, —NRC(O)—, —C(O)O—, and —NRC(O)NR;
V¹ is absent or is selected from ¹²C, ¹³C, H, D, halo, —CN, C₁₋₆ alkyl, monosaccharides, disaccharides, oligosaccharides, polysaccharides, polyethylene glycols comprising repeating units of (OCH₂CH₂)ₙ, where n=1-3000;
V² is selected from heterocycloalkyl rings, heteroaryl rings, ¹²C, ¹³C, H, D, B, halo, —CN, C₁₋₆ alkyl, monosaccharides, disaccharides, oligosaccharides, polysaccharides, polyethylene glycols comprising repeating units of (OCH₂CH₂)ₙ, where n=1-3000, C₂₋₆ alkenyl, C₁₋₆ haloalkyl, —NRR, —OR, wherein the heteroaryl rings are 5- or 6-membered, the heterocycloalkyl rings are 3- to 8-membered, and wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴; and cyclo-alkyls (C₁₋₁₀);
ring A is selected from aromatic, heteroaryl, and cycloaryl rings, wherein the aromatic and heteroaryl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cycloalkyls (C₁₋₁₀), and wherein the cycloalkyl ring is an unsubstituted heterocycloalkyl ring or a heterocycloalkyl ring substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cyclo-alkyls (C₁₋₁₀);
ring B is selected from heteroaryl, aromatic, and cycloaryl rings, wherein the aromatic and heteroaryl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cycloalkyls (C₁₋₁₀), and wherein the cycloalkyl ring is an unsubstituted heterocycloalkyl ring or a heterocycloalkyl ring substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cyclo-alkyls (C₁₋₁₀); and
R is selected from H, D, B, halo, —CN and C₁₋₆ alkyl, C₂₋₆ alkenyl, C₁₋₆ haloalkyl, monosaccharides, disaccharides, oligosaccharides, polysaccharides, polyethylene glycols comprising repeating units of (OCH₂CH₂)ₙ, where n=1-3000, OR¹, OR⁴, NR¹R¹, NR⁴R⁴, NR¹R⁴, 5- or 6-membered heteroaryl rings, and 3- to 8-membered heterocycloalkyl rings, wherein the heteroaryl and heterocycloalkyl rings are unsubstituted, or are substituted with 1-4 groups selected from: oxo, halo, OR¹, OR⁴, NR¹R¹, NR⁴R⁴, NR¹R⁴, C₁₋₆ alkyl, C₁₋₆ alkyl substituted with OR¹, OR⁴, C₁₋₆ alkyl substituted with NR¹R¹, NR⁴R⁴, or NR¹R⁴, C₁₋₆ alkyl substituted with —C(O)R¹, —C(O)R⁴, —NR¹C(O)R¹, —NR⁴C(O)R⁴, NRIC(O)R⁴, or —NR⁴C(O)R¹; and cycloalkyls (C₁₋₁₀).

In certain embodiments of the above Formula V, when Z¹ is H, at least one of R³ and R⁴ is not H or D, or when Z¹ or Z² comprise a P, at least one of R³ and R⁴ is not H or D.

In particular embodiments, the present disclosure is directed to glycoconjugates comprising DMX-5804 or analogs thereof having a carbohydrate moiety linked thereto. For example, Scheme 3 shows such a glycoconjugate, wherein R is a monosaccharide. Similarly, in compounds having chemical structure I, the carbohydrate is linked as group R. In analog compounds having the structure of Formulas IV or V, the carbohydrate may be linked as at least one of the R groups R¹, R², V¹, or V². The carbohydrate moiety may be a monosaccharide, a disaccharide (two monosaccharides linked together), an oligosaccharide (three or more (e.g., 3-10) monosaccharides liked together), a polysaccharide (a polymer comprising ten or more linked monosaccharides), a glycosylamine (amino sugar), an alcohol, an alditol, a glycol, or a polyol. Examples of monosaccharides (e.g., pentoses and hexoses), disaccharides, oligosaccharides, polysaccharides, alcohols, alditols, glycols, and/or polyols, include, but are not limited to, erythulose, arabinose, 2,2-Bis[4(2,3-hydroxypropoxy)phenyl]propane (bis-HPPP), cellobiose, mannitol, mannose, glucose, ribose, allose, altrose, gulose, idose, lactose, maltose, dextrose, galactose, talose, psicose, fructose, sorbose, tagatose, β-d-ribopyranose, U-d-ribopyranose, β-d-ribofuranose, u-d-ribofuranose, sucrose, xylose, trehalose, raffinose, stachyose, sucralose, isomalt, isomaltulose, maltodextrins, fructo-oligosaccharides, amylose, amylopectin, starch, glycogen, cellulose, hemicellulose, methyl cellulose, methyl ethyl cellulose, pectins, hydrocolloids, isomaltooligosaccharide, maltodextrin, and/or polydextrose, glucosamine, galactosamine, N-acetyl glucosamine, N-acetyl galactosamine, mannosamine, neuraminic acid, bacillosamine, cyclitol, acarviocin, aminocyclitol, bornesitol, ciceritol, conduritol, decahydroxycyclopentane, 5-deoxyinositol, dodecahydroxycyclohexane, ononitol, pinitol, pinpollitol, quebrachitol, theogallin, 3,4,5-tri-O-galloylquinic acid, inositol, inositol pentakisphosphate, cis-inositol, D-chiro-inositol, L-chiro-inositol, epi-inositol, neo-inositol, muco-inositol, neo-inositol, scyllo-inositol, sorbitol, threitol, arabitol, galactitol, iditol, volemitol, sorbitol, fucitol, xylitol, lactitol, erythritol, lactitol, maltitol, phytic acid, quinic acid, 2-methoxyethan-1-ol (methyl glycol), propylene glycol, 1,2-propanediol, ethylene glycol, low molecular weight polyethylene glycols (e.g., C2-C10), vegetable glycerine, dipropylene glycol, glycerol, panthenol, cytosine glycol, cyclohexane-1,2-diol, aminomethanol, ethyleneglycol, 1,3-propanediol, 1,4-butanediol, 2,2-dimethyl-1-butanol, ethanol, propanol, butanol, pentanol, hexanol, ethynol, acetylenediol, fenticlor, fucitol, gluconic acid, glucic acid, 2-heptanol, 3-heptanol, 2-hexanol, 3-hexanol, ribitol, ethylhexylglycerin, octoxyglycerin, glucuronic acid, glyceraldehyde, glyceric acid, glycerol 3-phosphate, glycerol monostearate, 2-octanediol, pinacol, racemic acid, tartaric acid, uronic acid, xylosan, 1,2-butanediol, 1,3-butanediol, 1,4-butanediol, 2,3-butanediol, 2-methyl-2,4-pentanediol, neopentyl glycol, and combinations thereof.

In summary, the present disclosure includes, in a non-limiting embodiment, a method for synthesizing a pyrrolo[2,3-d]pyrimidin-4-one compound, the method comprising the steps of:
(1) contacting a substituted phenyl analog with a phenylamine compound in the presence of an effective amount of a mild base under reaction conditions sufficient to make 1-(4-(2-methoxyethoxy)phenyl)-2-(phenylamino)ethan-1-one;
(2) reacting the 1-(4-(2-methoxyethoxy)phenyl)-2-(phenylamino)ethan-1-one with malononitrile in the presence of an effective amount of a mild base under reaction conditions sufficient to make 2-amino-4-(4-(2-methoxyethoxy)phenyl)-1-phenyl-1H-pyrrole-3-carbonitrile; and
(3) refluxing the 2-amino-4-(4-(2-methoxyethoxy)phenyl)-1-phenyl-1H-pyrrole-3-carbonitrile in a medium under reaction conditions for a duration of time sufficient to make 5-(4-(2-methoxyethoxy)phenyl)-7-phenyl-3,4a,7,7a-tetrahydro-4H-pyrrolo[2,3-d]pyrimidin-4-one (DMX-5804). In step (1) the substituted phenyl analog may be a substituted phenylacyl analog, which may be a substituted phenylacyl halide.

The substituted phenylacyl halide may be 2-halo-1-(4-(2-methoxyethoxy)phenyl)ethan-1-one. The halo of 2-halo-1-(4-(2 methoxyethoxy)phenyl)ethan-1-one may be selected from the group consisting of Br, Cl, F, and I. The substituted phenyl analog may be a substituted phenylacetic acid analog. The substituted phenyl analog may have the chemical structure II:

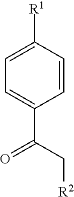

wherein R₁ of chemical structure II is selected from OH, OCH₃, H, OCH₂OH, COOH, NO₂, O-benzyl, O-substituted benzyl, O-esters poly(ethylene glycol)s (PEG), carbohydrates, amino acids, peptides, nucleobases, nucleosides, nucleotides, and oligonucleotides; and R₂ is selected from Cl, Br, F, I OH, NH₂, and p-Toluene sulfonylchlorides. In step (1) the phenylamine compound may be aniline or a substituted aniline. In step (1) the phenylamine compound may benzylamine or a benzylamine analog. In step (1) the phenylamine analog may have the following chemical structure III:

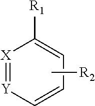

wherein R₁ of chemical structure III is selected from NH₂, Cl, Br, F, I, and CH₂NH₂, R₂ is selected from H, OH, OCH₃, NH₂, Cl, Br, F, I, NO₂, COOH, and SO₂NH₂, and X and Y are independently selected from C, N, and H. In the phenylamine compound one or both of R₁ and R₂ may be NH₂. In the phenylamine compound one, two, three, or four of R₁, R₂, X, and Y may comprise N. In step (1) the base may be selected from the group consisting of inorganic bases and organic bases. The inorganic base may be selected from the group consisting of NaHCO₃, Na₂CO₃, KHCO₃, and K₂CO₃. The organic base may be selected from diisopropylethylamine, trimethylamine, triethylamine, triethanolamine, potassium-tert-butoxide, and pyridine. In step (2) the base may be selected from the group consisting of, KOH, sodium hydroxide, lithium hydroxide, NaHCO₃, Na₂CO₃, KHCO₃, K₂CO₃, and organic bases, and the organic base may be selected from diisopropylethylamine, trimethylamine, triethylamine, triethanolamine, potassium-tert-butoxide, and pyridine. In steps (1) and (2), the base may have a pH in a range of 7.2 to 14. In steps (1) and (2), the base may have a pH in a range of 9 to 12. Each of steps (1) and (2) may be conducted at a temperature in a range of 25° C. to 150° C. Step (1) may conducted at a temperature in a range of 60° C. to 90° C. Step (2) may be conducted at a temperature in a range of 65° C. to 70° C. Step (1) may be conducted for a duration of time in a range of 2 h to 16 h, or in a range of 5 h to 12 h. Step (2) may be conducted for a duration of time in a range of 1 h to 20 h, or in a range of 6 h to 8 h. In step (1), the phenylamine compound may be dissolved in an organic solvent. The organic solvent may be selected from the group consisting of dimethylformamide (DMF), ethanol, methanol, acetonitrile, dichloromethane, acetone, tetrahydrofuran, toluene, dimethylsulfoxide (DMSO), hexane, ethyl acetate, and combinations thereof in any proportion. The 1-(4-(2-methoxyethoxy)phenyl)-2-(phenylamino)ethan-1-one may be recrystallized before it is used in step (2). In step (2) the 2-amino-4-(4-(2-methoxyethoxy)phenyl)-1-phenyl-1H-pyrrole-3-carbonitrile may be refluxed in a solvent selected from the group consisting of dimethylformamide (DMF), ethanol, methanol, acetonitrile, dichloromethane, acetone, tetrahydrofuran, toluene, dimethylsulfoxide (DMSO), hexane, ethyl acetate, and combinations thereof in any proportion. Step (3) may be conducted at a temperature in a range of 30° C. to 120° C. or in a range of 60° C. to 90° C. The refluxing in step (3) may be in a range of 2 h to 24 h, or in a range of 8 h to 16 h. The medium used in step (3) may be selected from the group consisting of formic acid, formaldehyde, formamidine, and salts thereof, and compounds having the structure RCHO, RCOOH, and RCONH₂, where R is hydrogen, alkyl or aryl. In step (3) the medium may be formic acid.

In another non-limiting embodiment, the present disclosure includes a method for synthesizing a pyrrolo[2,3-d]pyrimidin-4-one compound, the method comprising the steps of:
(1) contacting a substituted phenyl analog with a phenylamine compound and a solvent in the presence of an effective amount of a mild base under reaction conditions sufficient to make 1-(4-(2-methoxyphenyl)-2-(phenylamino)ethan-1-one;
(2) reacting the 1-(4-(2-methoxyphenyl)-2-(phenylamino)ethan-1-one with malononitrile in the presence of an effective amount of a mild base under reaction conditions sufficient to make 2-amino-4-(4-(2-methoxyphenyl)-1-phenyl-1H-pyrrole-3-carbonitrile;
(3) combining the 2-amino-4-(4-(2-methoxyphenyl)-1-phenyl-1H-pyrrole-3-carbonitrile with a strong Lewis acid in a solvent to form a mixture, and refluxing the mixture in a medium under reaction conditions for a duration of time sufficient to make 5-(4-(2-hydroxyphenyl)-7-phenyl-3,4a,7,7a-tetrahydro-4H-pyrrolo[2,3-d]pyrimidin-4-one; and
(4) combining the 5-(4-(2-hydroxyphenyl)-7-phenyl-3,4a,7,7a-tetrahydro-4H-pyrrolo[2,3-d]pyrimidin-4-one with boron trifluoride diethyl etherate and an R group-contributing substrate to form a mixture, and refluxing the mixture in a medium under reaction conditions for a duration of time sufficient to make an R group-oxyphenyl pyrrolo[2,3-d]pyrimidin-4-one compound.

In step (1) the substituted phenyl analog may be a substituted phenylacyl analog, which may be a substituted phenylacyl halide. The substituted phenylacyl halide may be 2-halo-1-(4-(2-methoxyethoxy)phenyl)ethan-1-one. The halo of 2-halo-1-(4-(2 methoxyethoxy)phenyl)ethan-1-one may be selected from the group consisting of Br, Cl, F, and I. The substituted phenyl analog may be a substituted phenylacetic acid analog. The substituted phenyl analog may have the chemical structure II:

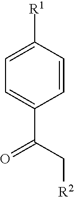

wherein R₁ is selected from OH, OCH₃, H, OCH₂OH, COOH, NO₂, O-benzyl, O-substituted benzyl, O-esters poly(ethylene glycol)s (PEG), carbohydrates, amino acids, peptides, nucleobases, nucleosides, nucleotides, and oligonucleotides; and R₂ is selected from Cl, Br, F, I OH, NH₂, and p-Toluene sulfonylchlorides. In step (1) the phenylamine compound may be aniline or a substituted aniline. In step (1) the phenylamine compound may benzylamine or a benzylamine analog. In step (1) the phenylamine compound may have the chemical structure III:

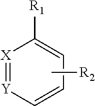

wherein R₁ is selected from NH₂, Cl, Br, F, I, and CH₂NH₂, R₂ is selected from H, OH, OCH₃, NH₂, Cl, Br, F, I, NO₂, COOH, and SO₂NH₂, and X and Y are selected from C, N, and H. In the phenylamine compound one or more of R₁ and R₂ may be NH₂. In the phenylamine compound one or more of R₁, R₂, X, and Y may comprise N. In step (1) the solvent may be selected from the group consisting of dimethylformamide (DMF), dimethylacetamide, formamide, N-formylmorpholine, N-Methyl-2-pyrrolidone, N-Methylformamide, 2-Pyrrolidone, tetramethyl urea, N-Vinylacetamide, N-Vinylpyrrolidone, and ethanol. In step (1) the base may be selected from the group consisting of inorganic bases and organic bases. The inorganic base may be selected from the group consisting of NaHCO₃, Na₂CO₃, KHCO₃, and K₂CO₃. The organic base may be selected from diisopropylethylamine, trimethylamine, triethylamine, triethanolamine, potassium-tert-butoxide, and pyridine. In step (2) the base may be selected from the group consisting of, KOH, sodium hydroxide, lithium hydroxide, NaHCO₃, Na₂CO₃, KHCO₃, K₂CO₃, and organic bases, and the organic base may be selected from diisopropylethylamine, trimethylamine, triethylamine, triethanolamine, potassium-tert-butoxide, and pyridine. In steps (1) and (2), the base may have a pH in a range of 7.2 to 14. In steps (1) and (2), the base may have a pH in a range of 9 to 12. Each of steps (1) and (2) may be conducted at a temperature in a range of 25° C. to 150° C. Step (1) may conducted at a temperature in a range of 60° C. to 90° C. Step (2) may be conducted at a temperature in a range of 65° C. to 70° C. Step (1) may be conducted for a duration of time in a range of 2 h to 16 h, or in a range of 5 h to 12 h. Step (2) may be conducted for a duration of time in a range of 1 h to 20 h, or in a range of 6 h to 8 h. In step (1), the phenylamine compound may be dissolved in an organic solvent, and the organic solvent may be selected from the group consisting of dimethylformamide (DMF), ethanol, methanol, acetonitrile, dichloromethane, acetone, tetrahydrofuran, toluene, dimethylsulfoxide (DMSO), hexane, ethyl acetate, and combinations thereof in any proportion. The 1-(4-(2-methoxyethoxy)phenyl)-2-(phenylamino)ethan-1-one may be recrystallized before it is used in step (2). In step (2) the 2-amino-4-(4-(2-methoxyethoxy)phenyl)-1-phenyl-1H-pyrrole-3-carbonitrile may be refluxed in a solvent selected from the group consisting of dimethylformamide (DMF), ethanol, methanol, acetonitrile, dichloromethane, acetone, tetrahydrofuran, toluene, dimethylsulfoxide (DMSO), hexane, ethyl acetate, and combinations thereof in any proportion. Step (3) may be conducted at a temperature in a range of 30° C. to 120° C. Step (3) may be conducted at a temperature in a range of 60° C. to 90° C. The duration of time of the refluxing in step (3) may be in a range of 2 h to 24 h. The duration of time of the refluxing in step (3) may be in a range of 8 h to 16 h. The medium used in step (3) may be selected from the group consisting of formic acid, formaldehyde, formamidine, and salts thereof, and compounds having the structure RCHO, RCOOH, and RCONH₂, where R is hydrogen, alkyl or aryl. The strong Lewis acid used in step (3) may be selected from the group consisting of boron trihalides, aluminum trihalides, and trimethyl borane. The boron trihalide may be selected from the group consisting of boron tribromide, boron trifluoride, boron trichloride, and boron triiodide. The aluminum trihalide may be selected from the group consisting of aluminum trichloride, aluminum tribromide, aluminum trifluoride, and aluminum triiodide. In step (4) the R group-contributing substrate may be selected from the group consisting of carbohydrates, poly(ethyleneglycol) chains, amino acids, peptides, proteins, peptide aptamers, oligonucleotide aptamers, ribonucleic acids, deoxyribonucleic acids, peptide nucleic acids, nucleobases, nucleosides, nucleotides, phosphate moieties (—PO₄), and sulfonyl moieties (—SONH₂). Step (4) may be conducted at a temperature in a range of 30° C. to 120° C. Step (4) may be conducted at a temperature in a range of 60° C. to 90° C. The duration of time of the refluxing in step (4) may be in a range of 2 h to 24 h. The duration of time of the refluxing in step (4) may be in a range of 8 h to 16 h. The medium used in refluxing in step (4) may be selected from the group consisting of dimethylformmide, dimethylsulfoxide, acetonitrile, ethanol, or acetone.

While the present disclosure has been described in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the structures of the various components described herein, or the methods described herein without departing from the spirit and scope of the present disclosure.

1. A method for synthesizing a pyrrolo[2,3-d]pyrimidin-4-one compound, the method comprising the steps of:
(1) contacting a substituted phenylacyl halide with a phenylamine compound in the presence of an effective amount of a mild base under reaction conditions sufficient to make 1-(4-(2-methoxyethoxy)phenyl)-2-(phenylamino)ethan-1-one;
(2) reacting the 1-(4-(2-methoxyethoxy)phenyl)-2-(phenylamino)ethan-1-one with malononitrile in the presence of an effective amount of a mild base under reaction conditions sufficient to make 2-amino-4-(4-(2-methoxyethoxy)phenyl)-1-phenyl-1H-pyrrole-3-carbonitrile; and
(3) refluxing the 2-amino-4-(4-(2-methoxyethoxy)phenyl)-1-phenyl-1H-pyrrole-3-carbonitrile in a medium under reaction conditions for a duration of time sufficient to make 5-(4-(2-methoxyethoxy)phenyl)-7-phenyl-3,4a,7,7a-tetrahydro-4H-pyrrolo[2,3-d]pyrimidin-4-one (DMX-5804).

2. The method of claim 1, wherein in step (1) the substituted phenylacyl halide is 2-halo-1-(4-(2-methoxyethoxy)phenyl)ethan-1-one.

3. The method of claim 1, wherein in step (1) the phenylamine compound is aniline.

4. The method of claim 1, wherein the 1-(4-(2-methoxyethoxy)phenyl)-2-(phenylamino)ethan-1-one is recrystallized before it is used in step (2).

5. The method of claim 1, wherein in step (2) the 2-amino-4-(4-(2-methoxyethoxy)phenyl)-1-phenyl-1H-pyrrole-3-carbonitrile is refluxed in a solvent selected from the group consisting of dimethylformamide (DMF), ethanol, methanol, acetonitrile, dichloromethane, acetone, tetrahydrofuran, toluene, dimethylsulfoxide (DMSO), hexane, ethyl acetate, and combinations thereof in any proportion.

6. The method of claim 1, wherein the medium used in step (3) is selected from the group consisting of formic acid, formaldehyde, formamidine, and salts thereof, and compounds having the structure RCHO, RCOOH, and RCONH₂, where R is hydrogen, alkyl or aryl.

7. The method of claim 1, wherein in step (1), the phenylamine compound is dissolved in an organic solvent.

8. The method of claim 7, wherein the organic solvent is selected from the group consisting of dimethylformamide (DMF), ethanol, methanol, acetonitrile, dichloromethane, acetone, tetrahydrofuran, toluene, dimethylsulfoxide (DMSO), hexane, ethyl acetate, and combinations thereof in any proportion.

9. A method for synthesizing a pyrrolo[2,3-d]pyrimidin-4-one compound, the method comprising the steps of:
(1) contacting a substituted phenylacyl halide with a phenylamine compound and a solvent in the presence of an effective amount of a mild base under reaction conditions sufficient to make 1-(4-(2-methoxyphenyl)-2-(phenylamino)ethan-1-one;
(2) reacting the 1-(4-(2-methoxyphenyl)-2-(phenylamino)ethan-1-one with malononitrile in the presence of an effective amount of a mild base under reaction conditions sufficient to make 2-amino-4-(4-(2-methoxyphenyl)-1-phenyl-1H-pyrrole-3-carbonitrile;
(3) combining the 2-amino-4-(4-(2-methoxyphenyl)-1-phenyl-1H-pyrrole-3-carbonitrile with a strong Lewis acid in a solvent to form a mixture, and refluxing the mixture in a medium under reaction conditions for a duration of time sufficient to make 5-(4-(2-hydroxyphenyl)-7-phenyl-3,4a,7,7a-tetrahydro-4H-pyrrolo[2,3-d]pyrimidin-4-one; and
(4) combining the 5-(4-(2-hydroxyphenyl)-7-phenyl-3,4a,7,7a-tetrahydro-4H-pyrrolo[2,3-d]pyrimidin-4-one with boron trifluoride diethyl etherate and an R group-contributing substrate to form a mixture, and refluxing the mixture in a medium under reaction conditions for a duration of time sufficient to make an R group-oxyphenyl pyrrolo[2,3-d]pyrimidin-4-one compound.

10. The method of claim 9, wherein in step (1) the phenylamine compound is aniline or a substituted aniline.

11. The method of claim 9, wherein in step (1) the substituted phenylacyl halide is 2-halo-1-(4-(2-methoxyphenyl)ethan-1-one.

12. The method of claim 9, wherein in step (1) the solvent is selected from the group consisting of dimethylformamide (DMF), dimethylacetamide, formamide, N-formylmorpholine, N-Methyl-2-pyrrolidone, N-Methylformamide, 2-Pyrrolidone, tetramethyl urea, N-Vinylacetamide, N-Vinylpyrrolidone, and ethanol.

13. The method of claim 9, wherein the 1-(4-(2-methoxyphenyl)-2-(phenylamino)ethan-1-one is recrystallized before it is used in step (2).

14. The method of claim 9, wherein in step (2) the 2-amino-4-(4-(2-methoxyphenyl)-1-phenyl-1H-pyrrole-3-carbonitrile is refluxed in a solvent selected from the group consisting of dimethylformamide (DMF), ethanol, methanol, acetonitrile, dichloromethane, acetone, tetrahydrofuran, toluene, dimethylsulfoxide (DMSO), hexane, ethyl acetate, and combinations thereof.

15. The method of claim 9, wherein the medium used in step (3) is selected from the group consisting of formic acid, formaldehyde, formamidine, and salts thereof, and compounds having the structure RCHO, RCOOH, and RCONH₂, where R is hydrogen, alkyl or aryl.

16. The method of claim 9, wherein in step (3), the strong Lewis acid is selected from the group consisting of boron trihalides, aluminum trihalides, and trimethyl borane.

17. The method of claim 9, wherein in step (4) the R group-contributing substrate is selected from the group consisting of carbohydrates, poly(ethyleneglycol) chains, amino acids, peptides, proteins, peptide aptamers, oligonucleotide aptamers, ribonucleic acids, deoxyribonucleic acids, peptide nucleic acids, nucleobases, nucleosides, nucleotides, phosphate moieties (—PO₄), and sulfonyl moieties (—SONH₂).

18. The method of claim 9, wherein the medium used in refluxing in step (4) is selected from the group consisting of dimethylformmide, dimethylsulfoxide, acetonitrile, ethanol, or acetone.

19. A compound having formula (IV), or a pharmaceutically acceptable salt thereof: 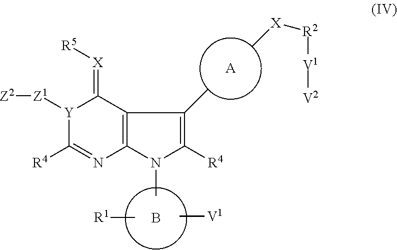
wherein:
X is selected from 0, OH, and S;
Y is selected from N and NH;
R¹ is selected from H, deuterium (D), halo, —CN and C₁₋₆ alkyl, monosaccharides, disaccharides, oligosaccharides, polysaccharides, and polyethylene glycols comprising repeating units of (OCH₂CH₂)ₙ, where n=1-3000;
R² is absent, or is selected from H, D, halo, —CN and C₁₋₆ alkyl, monosaccharides, disaccharides, oligosaccharides, polysaccharides, and polyethylene glycols comprising repeating units of (OCH₂CH₂)ₙ, where n=1-3000;
R³ is selected from H, borane (B), halo, C₁₋₆ alkyl, C₂₋₆ alkenyl, C1-₆ haloalkyl, —NRR, —OR, 5 or 6 membered heteroaryl rings, and 3 to 8 membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀);
R⁴ is selected from H, borane (B), halo, C₁₋₆ alkyl, C₂₋₆ alkenyl, C₁₋₆ haloalkyl, —NRR, —OR, 5- or 6-membered heteroaryl rings, and 3- to 8-membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀);
R⁵ is selected from H, borane (B), halo, C₁₋₆ alkyl, C₂₋₆ alkenyl, C₁₋₆ haloalkyl, —NRR, —OR, P(O)(OH)₂, P(O)(O—C₁₋₆alkyl))₂, 5- or 6-membered heteroaryl rings, and 3- to 8-membered heterocycloalkyl rings, wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, —NR⁴C(O)R⁴; and cycloalkyls (C₁₋₁₀), or is absent when X is O;
Z¹ is selected from H, C, O, P, C₁₋₁₀ alkyl, phenyl, substituted phenyl, and benzyl;
Z² is absent or is selected from —NR—, —O—, —C(O)—, —SO₂—, —SO₂NR—, —NRSO₂—, —C(O)NR—, —NRC(O)—, —C(O)O—, and —NRC(O)NR;
V¹ is absent or is selected from ¹²C, ¹³C, H, D, halo, —CN, C₁₋₆ alkyl, monosaccharides, disaccharides, oligosaccharides, polysaccharides, polyethylene glycols comprising repeating units of (OCH₂CH₂)ₙ, where n=1-3000;
V² is selected from monosaccharides, heterocycloalkyl rings, heteroaryl rings, ¹²C, ¹³C, H, D, B, halo, —CN, C₁₋₆ alkyl, disaccharides, oligosaccharides, polysaccharides, polyethylene glycols comprising repeating units of (OCH₂CH₂)ₙ, where n=1-3000, C₂₋₆ alkenyl, C₁₋₆ haloalkyl, —NRR, —OR, wherein the heteroaryl rings are 5- or 6-membered, the heterocycloalkyl rings are 3- to 8-membered, and wherein the heteroaryl and heterocycloalkyl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴; and cyclo-alkyls (C₁₋₁₀);
ring A is selected from aromatic, heteroaryl, and cycloaryl rings, wherein the aromatic and heteroaryl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cycloalkyls (C₁₋₁₀), and wherein the cycloalkyl ring is an unsubstituted heterocycloalkyl ring or a heterocycloalkyl ring substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cyclo-alkyls (C₁₋₁₀);
ring B is selected from heteroaryl, aromatic, and cycloaryl rings, wherein the aromatic and heteroaryl rings are unsubstituted or substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cycloalkyls (C₁₋₁₀), and wherein the cycloalkyl ring is an unsubstituted heterocycloalkyl ring or a heterocycloalkyl ring substituted with 1-4 groups selected from: oxo, halo, OR, C₁₋₆ alkyl, C₁₋₆ alkyl substituted with NRR¹, C₁₋₆ alkyl substituted with OR¹, —C(O)R⁴, and —NR⁴C(O)R⁴, and cyclo-alkyls (C₁₋₁₀); and
R is selected from H, D, B, halo, —CN and C₁₋₆ alkyl, C₂₋₆ alkenyl, C₁₋₆ haloalkyl, monosaccharides, disaccharides, oligosaccharides, polysaccharides, polyethylene glycols comprising repeating units of (OCH₂CH₂)ₙ, where n=1-3000, OR¹, OR⁴, NR¹R¹, NR⁴R⁴, NR¹R⁴, 5- or 6-membered heteroaryl rings, and 3- to 8-membered heterocycloalkyl rings, wherein the heteroaryl and heterocycloalkyl rings are unsubstituted, or are substituted with 1-4 groups selected from: oxo, halo, OR¹, OR⁴, N¹R¹, NR⁴R⁴, NR¹R⁴, C₁₋₆ alkyl, C₁₋₆ alkyl substituted with OR¹, OR⁴, C₁₋₆ alkyl substituted with NR¹R¹, NR⁴R⁴, or NR¹R⁴, C₁₋₆ alkyl substituted with —C(O)R¹, —C(O)R⁴, —NR¹C(O)R¹, —NR⁴C(O)R⁴, NR¹C(O)R⁴, or —NR⁴C(O)R¹; and cycloalkyls (C₁₋₁₀).

20. The compound of claim 19, wherein when Z¹ is H, at least one of R³ and R⁴ is not H or D, or when Z¹ or Z² comprise a P, at least one of R³ and R⁴ is not H or D.

21. The compound of claim 19, wherein X═O, Y═NH, R¹═H or D, R² is absent, R³═H, R⁴═H, R⁵ is absent, Z¹ is absent, Z² is absent, ring A is an aromatic ring, ring B is an unsubstituted aromatic ring, V¹ is absent, and V² is a monosaccharide.

22. The compound of claim 21, wherein V² is selected from the group consisting of glucose, ribose, galactose, sorbose, mannose, fructose, tagatose, allose, altrose, gulose, idose, talose, and psicose.

23. The method of claim 9, wherein in step (4) the R group of the R group-oxyphenyl pyrrolo[2,3-d]pyrimidin-4-one compound is selected from the group consisting of glucose, ribose, galactose, sorbose, mannose, fructose, tagatose, allose, altrose, gulose, idose, talose, and psicose.
